(19) United States
(12) Patent Application Publication
(10) Pub. No.: US 2021/0403022 A1
(43) Pub. Date: Dec. 30, 2021

(54) METHOD FOR CONTROLLING VEHICLE AND INTELLIGENT COMPUTING APPARATUS CONTROLLING THE VEHICLE

(71) Applicant: LG ELECTRONICS INC., Seoul (KR)

(72) Inventors: Seungbum HONG, Seoul (KR); Beomoh KIM, Seoul (KR)

(73) Assignee: LG ELECTRONICS INC., Seoul (KR)

(21) Appl. No.: 16/495,376

(22) PCT Filed: Jul. 5, 2019
(86) PCT No.: PCT/KR2019/008316

§ 371 Date: Sep. 18, 2019

(51) Int. Cl. B60W 50/16; B60W 40/08; G06F 3/01; G06K 9/00; G06K 9/46; H04W 74/00; H04W 56/00; G06N 20/00

(52) U.S. Cl. CPC B60W 50/16; B60W 40/08; G06F 3/015; G06K 9/00845; B60W 2050/146; H04W 74/006; H04W 56/001; G06N 20/00; G06K 9/4661

(57) ABSTRACT

Disclosed are a method for controlling a vehicle and an intelligent computing apparatus for controlling a vehicle. The method for controlling a vehicle includes obtaining state information related to a driver in the vehicle, generating information related to concentration on the basis of the state information related to the driver, and outputting information related to drowsiness prevention on the basis of the information related to concentration, whereby it is possible to prevent driver's concentration from being decreased and it is possible to prevent occurrence of an accident due to carelessness such as decreased concentration of the driver during driving in advance by recognizing a state in which the driver's concentration is significantly decreased and providing information related to drowsiness prevention to increase concentration again. One or more of the vehicle, user terminal, and server of the present invention may be associated with an Artificial Intelligence Intelligenfce module, a robot, an Augmented Reality (AR) device, a Virtual Reality (VR) device, a device associated with a 5G service, or the like. Can be.

### TECHNICAL FIELD

The present invention relates to a vehicle control method and an intelligent computing device for controlling a vehicle, and more particularly, to a vehicle control method for reflecting driver's state information and an intelligent computing device for controlling a vehicle.

### BACKGROUND ART

Vehicles may be classified as internal combustion engine vehicles, external combustion engine vehicles, gas turbine vehicles, electric vehicles, and the like, according to types of prime movers used therein.

An autonomous vehicle refers to a vehicle which is operable by itself without a driver's or occupant's operation, and an automated vehicle & highway system refers to a system which monitors and controls such an autonomous vehicle to be operated by itself.

A vehicle is a means of transportation for moving a user therein in a desired direction, and a typical example thereof may be an automobile. While the vehicle provides a user with ease of movement, the user must watch the front and back of the vehicle carefully during driving. Here, the front and back may refer to an object, i.e., a driving obstructing element such as a person, a vehicle, an obstacle, and the like, which is close to the vehicle.

Meanwhile, in the related art, in order to escape from decreased concentration such as carelessness, drowsiness, or the like, the driver must directly take action to increase his or her concentration by directly regularly pressing a drowsiness preventing button, directly opening the windows for ventilation, and the like.

However, it may be difficult for the driver who has significantly decreased concentration to take action to directly increase his/her concentration, and currently, a technology for increasing driver's concentration in the vehicle itself is insufficient.

### DISCLOSURE

### Technical Problem

An object of the present disclosure is to meet the needs and solve the problems.

It is another object of the present disclosure to provide a method for controlling a vehicle for increasing driver's decreased concentration and an intelligent computing apparatus for controlling the vehicle.

### Technical Solution

According to an aspect of the present disclosure, there is provided a method for controlling a vehicle including: obtaining state information related to a driver in a vehicle; and outputting information related to drowsiness prevention on the basis of the state information related to the driver, wherein the outputting the information related to drowsiness prevention includes: generating information related to concentration of the driver by analyzing the state information related to the driver; and outputting the information related to drowsiness prevention on the basis of the information related to concentration.

The obtaining of the state information related to the driver may include detecting an electro-encephalography, EEG, signal of the driver.

The obtaining the state information related to the driver may include capturing an image of the driver, and the generating of the information related to concentration of the driver may include generating the information related to concentration on the basis of action information (or behavior information) of the captured image of the driver.

The outputting the information related to drowsiness prevention may include outputting light having a predetermined wavelength through illumination of the vehicle.

The outputting of the information related to drowsiness prevention may include outputting vibration through a seat of the vehicle.

The outputting of the information related to drowsiness prevention may include outputting information for guiding a road to a predetermined rest area through a display of the vehicle.

The method may further include: receiving, from a network, downlink control information (DCI) used for scheduling transmission of state information of the driver obtained from at least one sensor provided in the vehicle, wherein the state information of the driver is transmitted to the network on the basis of the DCI.

The method may further include: performing an initial access procedure with the network on the basis of a synchronization signal block, SSB, wherein the state information of the driver is transmitted to the network through a PUSCH, the SSB and a DM-RS of the PUSCH are quasi-co-located, QCL, for a QCL type D.

The method may further include: transmitting the state information of the driver to an artificial intelligence, AI, processor included in the network; and receiving AI-processed information from the AI processor, wherein the AI processed information is information determined as one of a low concentration state (low), a medium concentration state (medium), and a high concentration state (high).

According to another aspect of the present disclosure, an intelligent computing apparatus for controlling a vehicle includes: a camera included in the vehicle; a sensing unit; a processor; and a memory including an instruction executable by the processor, wherein the instruction enables obtaining of state information related to a driver in the vehicle and outputting information related to drowsiness prevention on the basis of the state information related to the driver, and the outputting of the state information related to the driver may include: generating information related to concentration of the driver by analyzing the state information related to the driver and outputting the information related to drowsiness prevention on the basis of the information related to concentration.

The processor may detect an EEG signal of the driver.

The processor may capture an image of the driver and may generate the information related to concentration on the basis of action information of the captured image of the driver.

The processor may output light having a predetermined wavelength through illumination of the vehicle.

The processor may output vibration through a seat of the vehicle.

The processor may output information guiding a route to a predetermined rest area through a display of the vehicle.

The processor may receive, from a network, downlink control information (DCI) used for scheduling transmission of state information of the driver obtained from at least one sensor provided in the vehicle, and the state information of the driver may be transmitted to the network on the basis of the DCI.

The processor may perform an initial access procedure with the network on the basis of a synchronization signal block, SSB, the state information of the driver may be transmitted to the network through a PUSCH, and the SSB and a DM-RS of the PUSCH may be quasi-co-located, QCL, for a QCL type D.

The processor may control a communication unit to transmit the state information of the driver to a AI processor included in the network and control the communication unit to receive the AI-processed information from the AI processor, and the AI-processed information may be information determined as one of a low concentration state (low), a medium concentration state (medium), and a high concentration state (high).

### Advantageous Effects

The effects of the vehicle control method, vehicle and intelligent computing device controlling the vehicle according to the present invention will be described as follows.

According to the present disclosure, by continuously monitoring a state of a driver while on the move (i.e., during driving) and providing an interface increasing the driver's concentration on the basis of a monitoring result, it is possible to prevent the driver's concentration from being decreased.

Further, according to the present disclosure, by detecting state information of a driver through a camera or a hair band provided in a vehicle and accurately determining a level of driver's concentration, it is possible to recognize in advance a state in which the driver's concentration is significantly decreased and provide information related to drowsiness prevention for increasing concentration, thus preventing occurrence of an accident due to carelessness such as decreased concentration of the driver while on the move, or the like in advance.

### DESCRIPTION OF DRAWINGS

FIG. 1 is a block diagram of a wireless communication system to which methods proposed in the disclosure are applicable.

FIG. 2 shows an example of a signal transmission/reception method in a wireless communication system.

FIG. 3 shows an example of basic operations of an autonomous vehicle and a 5G network in a 5G communication system.

FIG. 4 shows an example of a basic operation between vehicles using 5G communication.

FIG. 5 illustrates a vehicle according to an embodiment of the present disclosure.

FIG. 6 is a control block diagram of the vehicle according to an embodiment of the present disclosure.

FIG. 7 is a control block diagram of an autonomous device according to an embodiment of the present disclosure.

FIG. 8 is a diagram showing a signal flow in an autonomous vehicle according to an embodiment of the present disclosure.

FIG. 9 is a diagram illustrating the interior of a vehicle according to an embodiment of the present disclosure.

FIG. 10 is a block diagram referred to in description of a cabin system for a vehicle according to an embodiment of the present disclosure.

FIG. 11 is a diagram referred to in description of a usage scenario of a user according to an embodiment of the present disclosure.

FIGS. 12 to 14 illustrate an electro-encephalography (EEG) detecting apparatus according to an embodiment of the present disclosure.

FIG. 15 is a block diagram illustrating an AI device.

FIG. 16 is a view illustrating a system in which an autonomous vehicle and an artificial intelligence, AI, device are linked according to an embodiment of the present disclosure.

FIG. 17 is a flowchart illustrating a vehicle control method according to an embodiment of the present disclosure.

FIG. 18 illustrates an example of obtaining state information of a driver in an embodiment of the present disclosure.

FIG. 19 illustrates an example of outputting information related to drowsiness prevention according to an embodiment of the present disclosure.

FIG. 20 illustrates another example of outputting information related to drowsiness prevention according to an embodiment of the present disclosure.

FIG. 21 illustrates another example of outputting information related to drowsiness prevention according to an embodiment of the present disclosure.

FIG. 22 illustrates another example of outputting information related to drowsiness prevention according to an embodiment of the present disclosure.

FIG. 23 illustrates another example of outputting information related to drowsiness prevention according to an embodiment of the present disclosure.

FIG. 24 is a view illustrating an example of determining a driver's concentration level in an embodiment of the present disclosure.

FIG. 25 illustrates an example of EEG changes on the basis of eye movement.

FIG. 26 illustrates an example of EEG modeling including both left/right brain wave information and eye movement signals.

FIG. 27 is a view illustrating another example of determining a drowsy state in an embodiment of the present disclosure.

Accompanying drawings included as a part of the detailed description for helping understand the present disclosure provide embodiments of the present disclosure and are provided to describe technical features of the present disclosure with the detailed description.

### MODE FOR INVENTION

Hereinafter, embodiments of the disclosure will be described in detail with reference to the attached drawings. The same or similar components are given the same reference numbers and redundant description thereof is omitted. The suffixes “module” and “unit” of elements herein are used for convenience of description and thus can be used interchangeably and do not have any distinguishable meanings or functions. Further, in the following description, if a detailed description of known techniques associated with the present disclosure would unnecessarily obscure the gist of the present disclosure, detailed description thereof will be omitted. In addition, the attached drawings are provided for easy understanding of embodiments of the disclosure and do not limit technical spirits of the disclosure, and the embodiments should be construed as including all modifications, equivalents, and alternatives falling within the spirit and scope of the embodiments.

While terms, such as “first”, “second”, etc., may be used to describe various components, such components must not be limited by the above terms. The above terms are used only to distinguish one component from another.

When an element is “coupled” or “connected” to another element, it should be understood that a third element may be present between the two elements although the element may be directly coupled or connected to the other element. When an element is “directly coupled” or “directly connected” to another element, it should be understood that no element is present between the two elements.

The singular forms are intended to include the plural forms as well, unless the context clearly indicates otherwise.

In addition, in the specification, it will be further understood that the terms “comprise” and “include” specify the presence of stated features, integers, steps, operations, elements, components, and/or combinations thereof, but do not preclude the presence or addition of one or more other features, integers, steps, operations, elements, components, and/or combinations.

A. Example of Block Diagram of UE and 5G Network

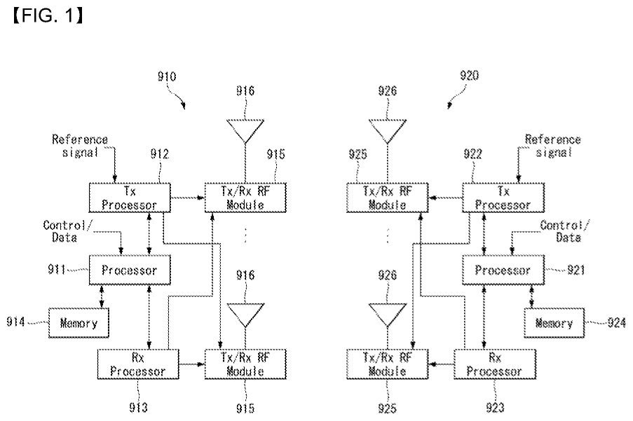

FIG. 1 is a block diagram of a wireless communication system to which methods proposed in the disclosure are applicable.

Referring to FIG. 1, a device (autonomous device) including an autonomous module is defined as a first communication device (910 of FIG. 1), and a processor 911 can perform detailed autonomous operations.

A 5G network including another vehicle communicating with the autonomous device is defined as a second communication device (920 of FIG. 1), and a processor 921 can perform detailed autonomous operations.

The 5G network may be represented as the first communication device and the autonomous device may be represented as the second communication device.

For example, the first communication device or the second communication device may be a base station, a network node, a transmission terminal, a reception terminal, a wireless device, a wireless communication device, an autonomous device, or the like.

For example, a terminal or user equipment (UE) may include a vehicle, a cellular phone, a smart phone, a laptop computer, a digital broadcast terminal, personal digital assistants (PDAs), a portable multimedia player (PMP), a navigation device, a slate PC, a tablet PC, an ultrabook, a wearable device (e.g., a smartwatch, a smart glass and a head mounted display (HMD)), etc. For example, the HMD may be a display device worn on the head of a user. For example, the HMD may be used to realize VR, AR or MR. Referring to FIG. 1, the first communication device 910 and the second communication device 920 include processors 911 and 921, memories 914 and 924, one or more Tx/Rx radio frequency (RF) modules 915 and 925, Tx processors 912 and 922, Rx processors 913 and 923, and antennas 916 and 926. The Tx/Rx module is also referred to as a transceiver. Each Tx/Rx module 915 transmits a signal through each antenna 926. The processor implements the aforementioned functions, processes and/or methods. The processor 921 may be related to the memory 924 that stores program code and data. The memory may be referred to as a computer-readable medium. More specifically, the Tx processor 912 implements various signal processing functions with respect to L1 (i.e., physical layer) in DL (communication from the first communication device to the second communication device). The Rx processor implements various signal processing functions of L1 (i.e., physical layer).

UL (communication from the second communication device to the first communication device) is processed in the first communication device 910 in a way similar to that described in association with a receiver function in the second communication device 920. Each Tx/Rx module 925 receives a signal through each antenna 926. Each Tx/Rx module provides RF carriers and information to the Rx processor 923. The processor 921 may be related to the memory 924 that stores program code and data. The memory may be referred to as a computer-readable medium.

B. Signal Transmission/Reception Method in Wireless Communication System

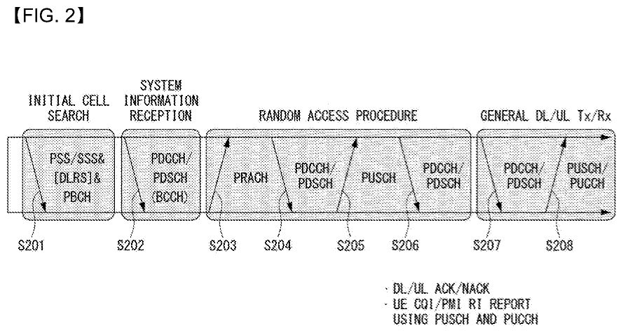

FIG. 2 is a diagram showing an example of a signal transmission/reception method in a wireless communication system.

Referring to FIG. 2, when a UE is powered on or enters a new cell, the UE performs an initial cell search operation such as synchronization with a BS (S201). For this operation, the UE can receive a primary synchronization channel (P-SCH) and a secondary synchronization channel (S-SCH) from the BS to synchronize with the BS and acquire information such as a cell ID. In LTE and NR systems, the P-SCH and S-SCH are respectively called a primary synchronization signal (PSS) and a secondary synchronization signal (SSS). After initial cell search, the UE can acquire broadcast information in the cell by receiving a physical broadcast channel (PBCH) from the BS. Further, the UE can receive a downlink reference signal (DL RS) in the initial cell search step to check a downlink channel state. After initial cell search, the UE can acquire more detailed system information by receiving a physical downlink shared channel (PDSCH) according to a physical downlink control channel (PDCCH) and information included in the PDCCH (S202).

Meanwhile, when the UE initially accesses the BS or has no radio resource for signal transmission, the UE can perform a random access procedure (RACH) for the BS (steps S203 to S206). To this end, the UE can transmit a specific sequence as a preamble through a physical random access channel (PRACH) (S203 and S205) and receive a random access response (RAR) message for the preamble through a PDCCH and a corresponding PDSCH (S204 and S206). In the case of a contention-based RACH, a contention resolution procedure may be additionally performed.

After the UE performs the above-described process, the UE can perform PDCCH/PDSCH reception (S207) and physical uplink shared channel (PUSCH)/physical uplink control channel (PUCCH) transmission (S208) as normal uplink/downlink signal transmission processes. Particularly, the UE receives downlink control information (DCI) through the PDCCH. The UE monitors a set of PDCCH candidates in monitoring occasions set for one or more control element sets (CORESET) on a serving cell according to corresponding search space configurations. A set of PDCCH candidates to be monitored by the UE is defined in terms of search space sets, and a search space set may be a common search space set or a UE-specific search space set. CORESET includes a set of (physical) resource blocks having a duration of one to three OFDM symbols. A network can configure the UE such that the UE has a plurality of CORESETs. The UE monitors PDCCH candidates in one or more search space sets. Here, monitoring means attempting decoding of PDCCH candidate(s) in a search space. When the UE has successfully decoded one of PDCCH candidates in a search space, the UE determines that a PDCCH has been detected from the PDCCH candidate and performs PDSCH reception or PUSCH transmission on the basis of DCI in the detected PDCCH. The PDCCH can be used to schedule DL transmissions over a PDSCH and UL transmissions over a PUSCH. Here, the DCI in the PDCCH includes downlink assignment (i.e., downlink grant (DL grant)) related to a physical downlink shared channel and including at least a modulation and coding format and resource allocation information, or an uplink grant (UL grant) related to a physical uplink shared channel and including a modulation and coding format and resource allocation information.

An initial access (IA) procedure in a 5G communication system will be additionally described with reference to FIG. 2.

The UE can perform cell search, system information acquisition, beam alignment for initial access, and DL measurement on the basis of an SSB. The SSB is interchangeably used with a synchronization signal/physical broadcast channel (SS/PBCH) block.

The SSB includes a PSS, an SSS and a PBCH. The SSB is configured in four consecutive OFDM symbols, and a PSS, a PBCH, an SSS/PBCH or a PBCH is transmitted for each OFDM symbol. Each of the PSS and the SSS includes one OFDM symbol and 127 subcarriers, and the PBCH includes 3 OFDM symbols and 576 subcarriers.

Cell search refers to a process in which a UE acquires time/frequency synchronization of a cell and detects a cell identifier (ID) (e.g., physical layer cell ID (PCI)) of the cell. The PSS is used to detect a cell ID in a cell ID group and the SSS is used to detect a cell ID group. The PBCH is used to detect an SSB (time) index and a half-frame.

There are 336 cell ID groups and there are 3 cell IDs per cell ID group. A total of 1008 cell IDs are present. Information on a cell ID group to which a cell ID of a cell belongs is provided/acquired through an SSS of the cell, and information on the cell ID among 336 cell ID groups is provided/acquired through a PSS.

The SSB is periodically transmitted in accordance with SSB periodicity. A default SSB periodicity assumed by a UE during initial cell search is defined as 20 ms. After cell access, the SSB periodicity can be set to one of {5 ms, 10 ms, 20 ms, 40 ms, 80 ms, 160 ms} by a network (e.g., a BS).

Next, acquisition of system information (SI) will be described.

SI is divided into a master information block (MIB) and a plurality of system information blocks (SIBs). SI other than the MIB may be referred to as remaining minimum system information. The MIB includes information/parameter for monitoring a PDCCH that schedules a PDSCH carrying SIB1 (SystemInformationBlock1) and is transmitted by a BS through a PBCH of an SSB. SIB1 includes information related to availability and scheduling (e.g., transmission periodicity and SI-window size) of the remaining SIBs (hereinafter, SIBx, x is an integer equal to or greater than 2). SiBx is included in an SI message and transmitted over a PDSCH. Each SI message is transmitted within a periodically generated time window (i.e., SI-window).

A random access (RA) procedure in a 5G communication system will be additionally described with reference to FIG. 2.

A random access procedure is used for various purposes. For example, the random access procedure can be used for network initial access, handover, and UE-triggered UL data transmission. A UE can acquire UL synchronization and UL transmission resources through the random access procedure. The random access procedure is classified into a contention-based random access procedure and a contention-free random access procedure. A detailed procedure for the contention-based random access procedure is as follows.

A UE can transmit a random access preamble through a PRACH as Msg1 of a random access procedure in UL. Random access preamble sequences having different two lengths are supported. A long sequence length 839 is applied to subcarrier spacings of 1.25 kHz and 5 kHz and a short sequence length 139 is applied to subcarrier spacings of 15 kHz, 30 kHz, 60 kHz and 120 kHz.

When a BS receives the random access preamble from the UE, the BS transmits a random access response (RAR) message (Msg2) to the UE. A PDCCH that schedules a PDSCH carrying a RAR is CRC masked by a random access (RA) radio network temporary identifier (RNTI) (RA-RNTI) and transmitted. Upon detection of the PDCCH masked by the RA-RNTI, the UE can receive a RAR from the PDSCH scheduled by DCI carried by the PDCCH. The UE checks whether the RAR includes random access response information with respect to the preamble transmitted by the UE, that is, Msg1. Presence or absence of random access information with respect to Msg1 transmitted by the UE can be determined according to presence or absence of a random access preamble ID with respect to the preamble transmitted by the UE. If there is no response to Msg1, the UE can retransmit the RACH preamble less than a predetermined number of times while performing power ramping. The UE calculates PRACH transmission power for preamble retransmission on the basis of most recent pathloss and a power ramping counter.

The UE can perform UL transmission through Msg3 of the random access procedure over a physical uplink shared channel on the basis of the random access response information. Msg3 can include an RRC connection request and a UE ID. The network can transmit Msg4 as a response to Msg3, and Msg4 can be handled as a contention resolution message on DL. The UE can enter an RRC connected state by receiving Msg4.

C. Beam Management (BM) Procedure of 5G Communication System

A BM procedure can be divided into (1) a DL MB procedure using an SSB or a CSI-RS and (2) a UL BM procedure using a sounding reference signal (SRS). In addition, each BM procedure can include Tx beam swiping for determining a Tx beam and Rx beam swiping for determining an Rx beam.

The DL BM procedure using an SSB will be described.

Configuration of a beam report using an SSB is performed when channel state information (CSI)/beam is configured in RRC_CONNECTED.
A UE receives a CSI-ResourceConfig IE including CSI-SSB-ResourceSetList for SSB resources used for BM from a BS. The RRC parameter “csi-SSB-ResourceSetList” represents a list of SSB resources used for beam management and report in one resource set. Here, an SSB resource set can be set as {SSBx1, SSBx2, SSBx3, SSBx4,}. An SSB index can be defined in the range of 0 to 63.
The UE receives the signals on SSB resources from the BS on the basis of the CSI-SSB-ResourceSetList.
When CSI-RS reportConfig with respect to a report on SSBRI and reference signal received power (RSRP) is set, the UE reports the best SSBRI and RSRP corresponding thereto to the BS. For example, when reportQuantity of the CSI-RS reportConfig IE is set to ‘ssb-Index-RSRP’, the UE reports the best SSBRI and RSRP corresponding thereto to the BS.

When a CSI-RS resource is configured in the same OFDM symbols as an SSB and ‘QCL-TypeD’ is applicable, the UE can assume that the CSI-RS and the SSB are quasi co-located (QCL) from the viewpoint of ‘QCL-TypeD’. Here, QCL-TypeD may mean that antenna ports are quasi co-located from the viewpoint of a spatial Rx parameter. When the UE receives signals of a plurality of DL antenna ports in a QCL-TypeD relationship, the same Rx beam can be applied.

Next, a DL BM procedure using a CSI-RS will be described.

An Rx beam determination (or refinement) procedure of a UE and a Tx beam swiping procedure of a BS using a CSI-RS will be sequentially described. A repetition parameter is set to ‘ON’ in the Rx beam determination procedure of a UE and set to ‘OFF’ in the Tx beam swiping procedure of a BS.

First, the Rx beam determination procedure of a UE will be described.
The UE receives an NZP CSI-RS resource set IE including an RRC parameter with respect to ‘repetition’ from a BS through RRC signaling. Here, the RRC parameter ‘repetition’ is set to ‘ON’.
The UE repeatedly receives signals on resources in a CSI-RS resource set in which the RRC parameter ‘repetition’ is set to ‘ON’ in different OFDM symbols through the same Tx beam (or DL spatial domain transmission filters) of the BS.
The UE determines an RX beam thereof.
The UE skips a CSI report. That is, the UE can skip a CSI report when the RRC parameter ‘repetition’ is set to ‘ON’.

Next, the Tx beam determination procedure of a BS will be described.
A UE receives an NZP CSI-RS resource set IE including an RRC parameter with respect to ‘repetition’ from the BS through RRC signaling. Here, the RRC parameter ‘repetition’ is related to the Tx beam swiping procedure of the BS when set to ‘OFF’.
The UE receives signals on resources in a CSI-RS resource set in which the RRC parameter ‘repetition’ is set to ‘OFF’ in different DL spatial domain transmission filters of the BS.
The UE selects (or determines) a best beam.
The UE reports an ID (e.g., CRI) of the selected beam and related quality information (e.g., RSRP) to the BS. That is, when a CSI-RS is transmitted for BM, the UE reports a CRI and RSRP with respect thereto to the BS.

Next, the UL BM procedure using an SRS will be described.
A UE receives RRC signaling (e.g., SRS-Config IE) including a (RRC parameter) purpose parameter set to ‘beam management” from a BS. The SRS-Config IE is used to set SRS transmission. The SRS-Config IE includes a list of SRS-Resources and a list of SRS-ResourceSets. Each SRS resource set refers to a set of SRS-resources.

The UE determines Tx beamforming for SRS resources to be transmitted on the basis of SRS-SpatialRelation Info included in the SRS-Config IE. Here, SRS-SpatialRelation Info is set for each SRS resource and indicates whether the same beamforming as that used for an SSB, a CSI-RS or an SRS will be applied for each SRS resource.
When SRS-SpatialRelationInfo is set for SRS resources, the same beamforming as that used for the SSB, CSI-RS or SRS is applied. However, when SRS-SpatialRelationInfo is not set for SRS resources, the UE arbitrarily determines Tx beamforming and transmits an SRS through the determined Tx beamforming.

Next, a beam failure recovery (BFR) procedure will be described.

In a beamformed system, radio link failure (RLF) may frequently occur due to rotation, movement or beamforming blockage of a UE. Accordingly, NR supports BFR in order to prevent frequent occurrence of RLF. BFR is similar to a radio link failure recovery procedure and can be supported when a UE knows new candidate beams. For beam failure detection, a BS configures beam failure detection reference signals for a UE, and the UE declares beam failure when the number of beam failure indications from the physical layer of the UE reaches a threshold set through RRC signaling within a period set through RRC signaling of the BS. After beam failure detection, the UE triggers beam failure recovery by initiating a random access procedure in a PCell and performs beam failure recovery by selecting a suitable beam. (When the BS provides dedicated random access resources for certain beams, these are prioritized by the UE). Completion of the aforementioned random access procedure is regarded as completion of beam failure recovery.

D. URLLC (Ultra-Reliable and Low Latency Communication)

URLLC transmission defined in NR can refer to (1) a relatively low traffic size, (2) a relatively low arrival rate, (3) extremely low latency requirements (e.g., 0.5 and 1 ms), (4) relatively short transmission duration (e.g., 2 OFDM symbols), (5) urgent services/messages, etc. In the case of UL, transmission of traffic of a specific type (e.g., URLLC) needs to be multiplexed with another transmission (e.g., eMBB) scheduled in advance in order to satisfy more stringent latency requirements. In this regard, a method of providing information indicating preemption of specific resources to a UE scheduled in advance and allowing a URLLC UE to use the resources for UL transmission is provided.

NR supports dynamic resource sharing between eMBB and URLLC. eMBB and URLLC services can be scheduled on non-overlapping time/frequency resources, and URLLC transmission can occur in resources scheduled for ongoing eMBB traffic. An eMBB UE may not ascertain whether PDSCH transmission of the corresponding UE has been partially punctured and the UE may not decode a PDSCH due to corrupted coded bits. In view of this, NR provides a preemption indication. The preemption indication may also be referred to as an interrupted transmission indication.

With regard to the preemption indication, a UE receives DownlinkPreemption IE through RRC signaling from a BS. When the UE is provided with DownlinkPreemption IE, the UE is configured with INT-RNTI provided by a parameter int-RNTI in DownlinkPreemption IE for monitoring of a PDCCH that conveys DCI format 2_1. The UE is additionally configured with a corresponding set of positions for fields in DCI format 2_1 according to a set of serving cells and positionInDCI by INT-ConfigurationPerServing Cell including a set of serving cell indexes provided by servingCellID, configured having an information payload size for DCI format 2_1 according to dci-Payloadsize, and configured with indication granularity of time-frequency resources according to timeFrequencySect.

The UE receives DCI format 2_1 from the BS on the basis of the DownlinkPreemption IE.

When the UE detects DCI format 2_1 for a serving cell in a configured set of serving cells, the UE can assume that there is no transmission to the UE in PRBs and symbols indicated by the DCI format 2_1 in a set of PRBs and a set of symbols in a last monitoring period before a monitoring period to which the DCI format 2_1 belongs. For example, the UE assumes that a signal in a time-frequency resource indicated according to preemption is not DL transmission scheduled therefor and decodes data on the basis of signals received in the remaining resource region.

E. mMTC (Massive MTC)

mMTC (massive Machine Type Communication) is one of 5G scenarios for supporting a hyper-connection service providing simultaneous communication with a large number of UEs. In this environment, a UE intermittently performs communication with a very low speed and mobility. Accordingly, a main goal of mMTC is operating a UE for a long time at a low cost. With respect to mMTC, 3GPP deals with MTC and NB (NarrowBand)-IoT.

mMTC has features such as repetitive transmission of a PDCCH, a PUCCH, a PDSCH (physical downlink shared channel), a PUSCH, etc., frequency hopping, retuning, and a guard period.

That is, a PUSCH (or a PUCCH (particularly, a long PUCCH) or a PRACH) including specific information and a PDSCH (or a PDCCH) including a response to the specific information are repeatedly transmitted. Repetitive transmission is performed through frequency hopping, and for repetitive transmission, (RF) retuning from a first frequency resource to a second frequency resource is performed in a guard period and the specific information and the response to the specific information can be transmitted/received through a narrowband (e.g., 6 resource blocks (RBs) or 1 RB).

F. Basic Operation Between Autonomous Vehicles Using 5G Communication

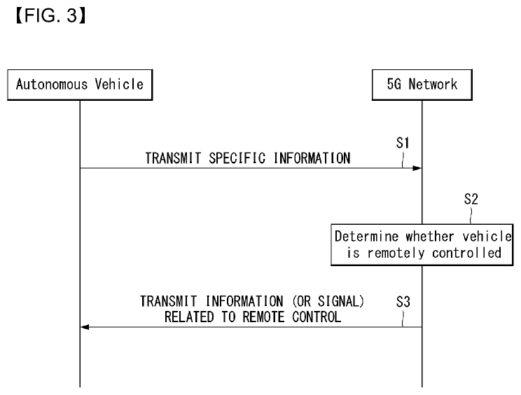

FIG. 3 shows an example of basic operations of an autonomous vehicle and a 5G network in a 5G communication system.

The autonomous vehicle transmits specific information to the 5G network (S1). The specific information may include autonomous driving related information. In addition, the 5G network can determine whether to remotely control the vehicle (S2). Here, the 5G network may include a server or a module which performs remote control related to autonomous driving. In addition, the 5G network can transmit information (or signal) related to remote control to the autonomous vehicle (S3).

G. Applied Operations Between Autonomous Vehicle and 5G Network in 5G Communication System

Hereinafter, the operation of an autonomous vehicle using 5G communication will be described in more detail with reference to wireless communication technology (BM procedure, URLLC, mMTC, etc.) described in FIGS. 1 and 2.

First, a basic procedure of an applied operation to which a method proposed by the present disclosure which will be described later and eMBB of 5G communication are applied will be described.

As in steps S1 and S3 of FIG. 3, the autonomous vehicle performs an initial access procedure and a random access procedure with the 5G network prior to step S1 of FIG. 3 in order to transmit/receive signals, information and the like to/from the 5G network.

More specifically, the autonomous vehicle performs an initial access procedure with the 5G network on the basis of an SSB in order to acquire DL synchronization and system information. A beam management (BM) procedure and a beam failure recovery procedure may be added in the initial access procedure, and quasi-co-location (QCL) relation may be added in a process in which the autonomous vehicle receives a signal from the 5G network.

In addition, the autonomous vehicle performs a random access procedure with the 5G network for UL synchronization acquisition and/or UL transmission. The 5G network can transmit, to the autonomous vehicle, a UL grant for scheduling transmission of specific information. Accordingly, the autonomous vehicle transmits the specific information to the 5G network on the basis of the UL grant. In addition, the 5G network transmits, to the autonomous vehicle, a DL grant for scheduling transmission of 5G processing results with respect to the specific information. Accordingly, the 5G network can transmit, to the autonomous vehicle, information (or a signal) related to remote control on the basis of the DL grant.

Next, a basic procedure of an applied operation to which a method proposed by the present disclosure which will be described later and URLLC of 5G communication are applied will be described.

As described above, an autonomous vehicle can receive DownlinkPreemption IE from the 5G network after the autonomous vehicle performs an initial access procedure and/or a random access procedure with the 5G network. Then, the autonomous vehicle receives DCI format 2_1 including a preemption indication from the 5G network on the basis of DownlinkPreemption IE. The autonomous vehicle does not perform (or expect or assume) reception of eMBB data in resources (PRBs and/or OFDM symbols) indicated by the preemption indication. Thereafter, when the autonomous vehicle needs to transmit specific information, the autonomous vehicle can receive a UL grant from the 5G network.

Next, a basic procedure of an applied operation to which a method proposed by the present disclosure which will be described later and mMTC of 5G communication are applied will be described.

Description will focus on parts in the steps of FIG. 3 which are changed according to application of mMTC.

In step S1 of FIG. 3, the autonomous vehicle receives a UL grant from the 5G network in order to transmit specific information to the 5G network. Here, the UL grant may include information on the number of repetitions of transmission of the specific information and the specific information may be repeatedly transmitted on the basis of the information on the number of repetitions. That is, the autonomous vehicle transmits the specific information to the 5G network on the basis of the UL grant. Repetitive transmission of the specific information may be performed through frequency hopping, the first transmission of the specific information may be performed in a first frequency resource, and the second transmission of the specific information may be performed in a second frequency resource. The specific information can be transmitted through a narrowband of 6 resource blocks (RBs) or 1 RB.

H. Autonomous Driving Operation Between Vehicles Using 5G Communication

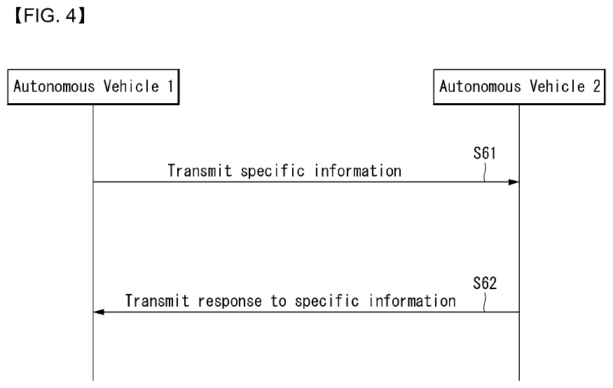

FIG. 4 shows an example of a basic operation between vehicles using 5G communication.

A first vehicle transmits specific information to a second vehicle (S61). The second vehicle transmits a response to the specific information to the first vehicle (S62).

Meanwhile, a configuration of an applied operation between vehicles may depend on whether the 5G network is directly (sidelink communication transmission mode 3) or indirectly (sidelink communication transmission mode 4) involved in resource allocation for the specific information and the response to the specific information.

Next, an applied operation between vehicles using 5G communication will be described.

First, a method in which a 5G network is directly involved in resource allocation for signal transmission/reception between vehicles will be described.

The 5G network can transmit DCI format 5A to the first vehicle for scheduling of mode-3 transmission (PSCCH and/or PSSCH transmission). Here, a physical sidelink control channel (PSCCH) is a 5G physical channel for scheduling of transmission of specific information a physical sidelink shared channel (PSSCH) is a 5G physical channel for transmission of specific information. In addition, the first vehicle transmits SCI format 1 for scheduling of specific information transmission to the second vehicle over a PSCCH. Then, the first vehicle transmits the specific information to the second vehicle over a PSSCH.

Next, a method in which a 5G network is indirectly involved in resource allocation for signal transmission/reception will be described.

The first vehicle senses resources for mode-4 transmission in a first window. Then, the first vehicle selects resources for mode-4 transmission in a second window on the basis of the sensing result. Here, the first window refers to a sensing window and the second window refers to a selection window. The first vehicle transmits SCI format 1 for scheduling of transmission of specific information to the second vehicle over a PSCCH on the basis of the selected resources. Then, the first vehicle transmits the specific information to the second vehicle over a PSSCH.

The above-described 5G communication technology can be combined with methods proposed in the present disclosure which will be described later and applied or can complement the methods proposed in the present disclosure to make technical features of the methods concrete and clear.

Driving

(1) Exterior of Vehicle

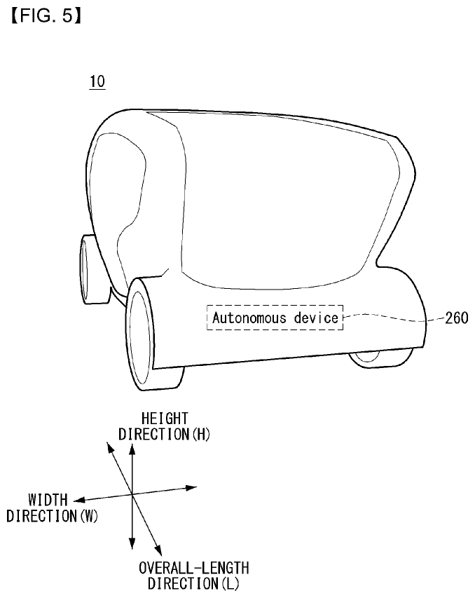

FIG. 5 is a diagram showing a vehicle according to an embodiment of the present disclosure.

Referring to FIG. 5, a vehicle 10 according to an embodiment of the present disclosure is defined as a transportation means traveling on roads or railroads. The vehicle 10 includes a car, a train and a motorcycle. The vehicle 10 may include an internal-combustion engine vehicle having an engine as a power source, a hybrid vehicle having an engine and a motor as a power source, and an electric vehicle having an electric motor as a power source. The vehicle 10 may be a private own vehicle. The vehicle 10 may be a shared vehicle. The vehicle 10 may be an autonomous vehicle.

(2) Components of Vehicle

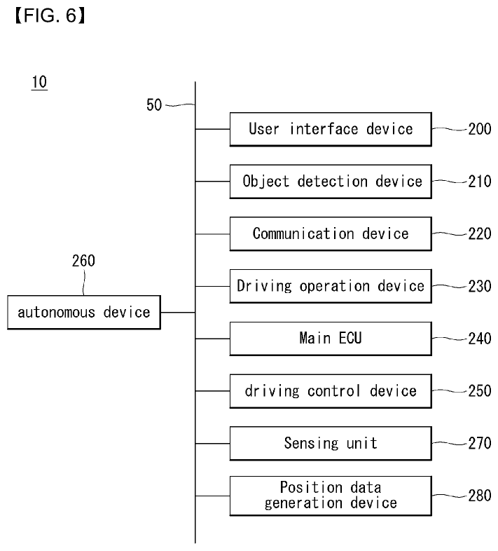

FIG. 6 is a control block diagram of the vehicle according to an embodiment of the present disclosure.

Referring to FIG. 6, the vehicle 10 may include a user interface device 200, an object detection device 210, a communication device 220, a driving operation device 230, a main ECU 240, a driving control device 250, an autonomous device 260, a sensing unit 270, and a position data generation device 280. The object detection device 210, the communication device 220, the driving operation device 230, the main ECU 240, the driving control device 250, the autonomous device 260, the sensing unit 270 and the position data generation device 280 may be realized by electronic devices which generate electric signals and exchange the electric signals from one another.

1) User Interface Device

The user interface device 200 is a device for communication between the vehicle 10 and a user. The user interface device 200 can receive user input and provide information generated in the vehicle 10 to the user. The vehicle 10 can realize a user interface (UI) or user experience (UX) through the user interface device 200. The user interface device 200 may include an input device, an output device and a user monitoring device.

2) Object Detection Device

The object detection device 210 can generate information about objects outside the vehicle 10. Information about an object can include at least one of information on presence or absence of the object, positional information of the object, information on a distance between the vehicle 10 and the object, and information on a relative speed of the vehicle 10 with respect to the object. The object detection device 210 can detect objects outside the vehicle 10. The object detection device 210 may include at least one sensor which can detect objects outside the vehicle 10. The object detection device 210 may include at least one of a camera, a radar, a lidar, an ultrasonic sensor and an infrared sensor. The object detection device 210 can provide data about an object generated on the basis of a sensing signal generated from a sensor to at least one electronic device included in the vehicle.

2.1) Camera

The camera can generate information about objects outside the vehicle 10 using images. The camera may include at least one lens, at least one image sensor, and at least one processor which is electrically connected to the image sensor, processes received signals and generates data about objects on the basis of the processed signals.

The camera may be at least one of a mono camera, a stereo camera and an around view monitoring (AVM) camera. The camera can acquire positional information of objects, information on distances to objects, or information on relative speeds with respect to objects using various image processing algorithms. For example, the camera can acquire information on a distance to an object and information on a relative speed with respect to the object from an acquired image on the basis of change in the size of the object over time. For example, the camera may acquire information on a distance to an object and information on a relative speed with respect to the object through a pin-hole model, road profiling, or the like. For example, the camera may acquire information on a distance to an object and information on a relative speed with respect to the object from a stereo image acquired from a stereo camera on the basis of disparity information.

The camera may be attached at a portion of the vehicle at which FOV (field of view) can be secured in order to photograph the outside of the vehicle. The camera may be disposed in proximity to the front windshield inside the vehicle in order to acquire front view images of the vehicle. The camera may be disposed near a front bumper or a radiator grill. The camera may be disposed in proximity to a rear glass inside the vehicle in order to acquire rear view images of the vehicle. The camera may be disposed near a rear bumper, a trunk or a tail gate. The camera may be disposed in proximity to at least one of side windows inside the vehicle in order to acquire side view images of the vehicle. Alternatively, the camera may be disposed near a side mirror, a fender or a door.

2.2) Radar

The radar can generate information about an object outside the vehicle using electromagnetic waves. The radar may include an electromagnetic wave transmitter, an electromagnetic wave receiver, and at least one processor which is electrically connected to the electromagnetic wave transmitter and the electromagnetic wave receiver, processes received signals and generates data about an object on the basis of the processed signals. The radar may be realized as a pulse radar or a continuous wave radar in terms of electromagnetic wave emission. The continuous wave radar may be realized as a frequency modulated continuous wave (FMCW) radar or a frequency shift keying (FSK) radar according to signal waveform. The radar can detect an object through electromagnetic waves on the basis of TOF (Time of Flight) or phase shift and detect the position of the detected object, a distance to the detected object and a relative speed with respect to the detected object. The radar may be disposed at an appropriate position outside the vehicle in order to detect objects positioned in front of, behind or on the side of the vehicle.

2.3) Lidar

The lidar can generate information about an object outside the vehicle 10 using a laser beam. The lidar may include a light transmitter, a light receiver, and at least one processor which is electrically connected to the light transmitter and the light receiver, processes received signals and generates data about an object on the basis of the processed signal. The lidar may be realized according to TOF or phase shift. The lidar may be realized as a driven type or a non-driven type. A driven type lidar may be rotated by a motor and detect an object around the vehicle 10. A non-driven type lidar may detect an object positioned within a predetermined range from the vehicle according to light steering. The vehicle 10 may include a plurality of non-drive type lidars. The lidar can detect an object through a laser beam on the basis of TOF (Time of Flight) or phase shift and detect the position of the detected object, a distance to the detected object and a relative speed with respect to the detected object. The lidar may be disposed at an appropriate position outside the vehicle in order to detect objects positioned in front of, behind or on the side of the vehicle.

3) Communication Device

The communication device 220 can exchange signals with devices disposed outside the vehicle 10. The communication device 220 can exchange signals with at least one of infrastructure (e.g., a server and a broadcast station), another vehicle and a terminal. The communication device 220 may include a transmission antenna, a reception antenna, and at least one of a radio frequency (RF) circuit and an RF element which can implement various communication protocols in order to perform communication.

For example, the communication device can exchange signals with external devices on the basis of C-V2X (Cellular V2X). For example, C-V2X can include sidelink communication based on LTE and/or sidelink communication based on NR. Details related to C-V2X will be described later.

For example, the communication device can exchange signals with external devices on the basis of DSRC (Dedicated Short Range Communications) or WAVE (Wireless Access in Vehicular Environment) standards based on IEEE 802.11p PHY/MAC layer technology and IEEE 1609 Network/Transport layer technology. DSRC (or WAVE standards) is communication specifications for providing an intelligent transport system (ITS) service through short-range dedicated communication between vehicle-mounted devices or between a roadside device and a vehicle-mounted device. DSRC may be a communication scheme that can use a frequency of 5.9 GHz and have a data transfer rate in the range of 3 Mbps to 27 Mbps. IEEE 802.11p may be combined with IEEE 1609 to support DSRC (or WAVE standards).

The communication device of the present disclosure can exchange signals with external devices using only one of C-V2X and DSRC. Alternatively, the communication device of the present disclosure can exchange signals with external devices using a hybrid of C-V2X and DSRC.

4) Driving Operation Device

The driving operation device 230 is a device for receiving user input for driving. In a manual mode, the vehicle 10 may be driven on the basis of a signal provided by the driving operation device 230. The driving operation device 230 may include a steering input device (e.g., a steering wheel), an acceleration input device (e.g., an acceleration pedal) and a brake input device (e.g., a brake pedal).

5) Main ECU

The main ECU 240 can control the overall operation of at least one electronic device included in the vehicle 10.

6) Driving Control Device

The driving control device 250 is a device for electrically controlling various vehicle driving devices included in the vehicle 10. The driving control device 250 may include a power train driving control device, a chassis driving control device, a door/window driving control device, a safety device driving control device, a lamp driving control device, and an air-conditioner driving control device. The power train driving control device may include a power source driving control device and a transmission driving control device. The chassis driving control device may include a steering driving control device, a brake driving control device and a suspension driving control device. Meanwhile, the safety device driving control device may include a seat belt driving control device for seat belt control.

The driving control device 250 includes at least one electronic control device (e.g., a control ECU (Electronic Control Unit)).

The driving control device 250 can control vehicle driving devices on the basis of signals received by the autonomous device 260. For example, the driving control device 250 can control a power train, a steering device and a brake device on the basis of signals received by the autonomous device 260.

7) Autonomous Device

The autonomous device 260 can generate a route for self-driving on the basis of acquired data. The autonomous device 260 can generate a driving plan for traveling along the generated route. The autonomous device 260 can generate a signal for controlling movement of the vehicle according to the driving plan. The autonomous device 260 can provide the signal to the driving control device 250.

The autonomous device 260 can implement at least one ADAS (Advanced Driver Assistance System) function. The ADAS can implement at least one of ACC (Adaptive Cruise Control), AEB (Autonomous Emergency Braking), FCW (Forward Collision Warning), LKA (Lane Keeping Assist), LCA (Lane Change Assist), TFA (Target Following Assist), BSD (Blind Spot Detection), HBA (High Beam Assist), APS (Auto Parking System), a PD collision warning system, TSR (Traffic Sign Recognition), TSA (Traffic Sign Assist), NV (Night Vision), DSM (Driver Status Monitoring) and TJA (Traffic Jam Assist).

The autonomous device 260 can perform switching from a self-driving mode to a manual driving mode or switching from the manual driving mode to the self-driving mode. For example, the autonomous device 260 can switch the mode of the vehicle 10 from the self-driving mode to the manual driving mode or from the manual driving mode to the self-driving mode on the basis of a signal received from the user interface device 200.

8) Sensing Unit

The sensing unit 270 can detect a state of the vehicle. The sensing unit 270 may include at least one of an internal measurement unit (IMU) sensor, a collision sensor, a wheel sensor, a speed sensor, an inclination sensor, a weight sensor, a heading sensor, a position module, a vehicle forward/backward movement sensor, a battery sensor, a fuel sensor, a tire sensor, a steering sensor, a temperature sensor, a humidity sensor, an ultrasonic sensor, an illumination sensor, and a pedal position sensor. Further, the IMU sensor may include one or more of an acceleration sensor, a gyro sensor and a magnetic sensor.

The sensing unit 270 can generate vehicle state data on the basis of a signal generated from at least one sensor. Vehicle state data may be information generated on the basis of data detected by various sensors included in the vehicle. The sensing unit 270 may generate vehicle attitude data, vehicle motion data, vehicle yaw data, vehicle roll data, vehicle pitch data, vehicle collision data, vehicle orientation data, vehicle angle data, vehicle speed data, vehicle acceleration data, vehicle tilt data, vehicle forward/backward movement data, vehicle weight data, battery data, fuel data, tire pressure data, vehicle internal temperature data, vehicle internal humidity data, steering wheel rotation angle data, vehicle external illumination data, data of a pressure applied to an acceleration pedal, data of a pressure applied to a brake panel, etc.

9) Position Data Generation Device

The position data generation device 280 can generate position data of the vehicle 10. The position data generation device 280 may include at least one of a global positioning system (GPS) and a differential global positioning system (DGPS). The position data generation device 280 can generate position data of the vehicle 10 on the basis of a signal generated from at least one of the GPS and the DGPS. According to an embodiment, the position data generation device 280 can correct position data on the basis of at least one of the inertial measurement unit (IMU) sensor of the sensing unit 270 and the camera of the object detection device 210. The position data generation device 280 may also be called a global navigation satellite system (GNSS).

The vehicle 10 may include an internal communication system 50. The plurality of electronic devices included in the vehicle 10 can exchange signals through the internal communication system 50. The signals may include data. The internal communication system 50 can use at least one communication protocol (e.g., CAN, LIN, FlexRay, MOST or Ethernet).

(3) Components of Autonomous Device

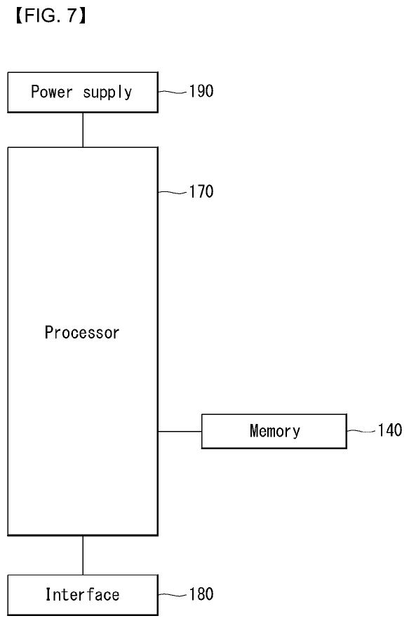

FIG. 7 is a control block diagram of the autonomous device according to an embodiment of the present disclosure.

Referring to FIG. 7, the autonomous device 260 may include a memory 140, a processor 170, an interface 180 and a power supply 190.

The memory 140 is electrically connected to the processor 170. The memory 140 can store basic data with respect to units, control data for operation control of units, and input/output data. The memory 140 can store data processed in the processor 170. Hardware-wise, the memory 140 can be configured as at least one of a ROM, a RAM, an EPROM, a flash drive and a hard drive. The memory 140 can store various types of data for overall operation of the autonomous device 260, such as a program for processing or control of the processor 170. The memory 140 may be integrated with the processor 170. According to an embodiment, the memory 140 may be categorized as a subcomponent of the processor 170.

The interface 180 can exchange signals with at least one electronic device included in the vehicle 10 in a wired or wireless manner. The interface 180 can exchange signals with at least one of the object detection device 210, the communication device 220, the driving operation device 230, the main ECU 240, the driving control device 250, the sensing unit 270 and the position data generation device 280 in a wired or wireless manner. The interface 180 can be configured using at least one of a communication module, a terminal, a pin, a cable, a port, a circuit, an element and a device.

The power supply 190 can provide power to the autonomous device 260. The power supply 190 can be provided with power from a power source (e.g., a battery) included in the vehicle 10 and supply the power to each unit of the autonomous device 260. The power supply 190 can operate according to a control signal supplied from the main ECU 240. The power supply 190 may include a switched-mode power supply (SMPS).

The processor 170 can be electrically connected to the memory 140, the interface 180 and the power supply 190 and exchange signals with these components. The processor 170 can be realized using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, and electronic units for executing other functions.

The processor 170 can be operated by power supplied from the power supply 190. The processor 170 can receive data, process the data, generate a signal and provide the signal while power is supplied thereto.

The processor 170 can receive information from other electronic devices included in the vehicle 10 through the interface 180. The processor 170 can provide control signals to other electronic devices in the vehicle 10 through the interface 180.

The autonomous device 260 may include at least one printed circuit board (PCB). The memory 140, the interface 180, the power supply 190 and the processor 170 may be electrically connected to the PCB.

(4) Operation of Autonomous Device

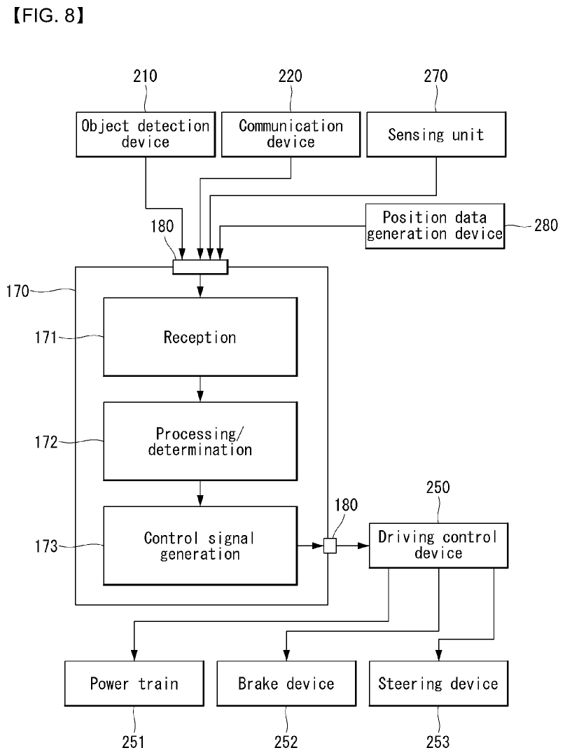

FIG. 8 is a diagram showing a signal flow in an autonomous vehicle according to an embodiment of the present disclosure.

1) Reception Operation

Referring to FIG. 8, the processor 170 can perform a reception operation. The processor 170 can receive data from at least one of the object detection device 210, the communication device 220, the sensing unit 270 and the position data generation device 280 through the interface 180. The processor 170 can receive object data from the object detection device 210. The processor 170 can receive HD map data from the communication device 220. The processor 170 can receive vehicle state data from the sensing unit 270. The processor 170 can receive position data from the position data generation device 280.

2) Processing/Determination Operation

The processor 170 can perform a processing/determination operation. The processor 170 can perform the processing/determination operation on the basis of traveling situation information. The processor 170 can perform the processing/determination operation on the basis of at least one of object data, HD map data, vehicle state data and position data.

2.1) Driving Plan Data Generation Operation

The processor 170 can generate driving plan data. For example, the processor 170 may generate electronic horizon data. The electronic horizon data can be understood as driving plan data in a range from a position at which the vehicle 10 is located to a horizon. The horizon can be understood as a point a predetermined distance before the position at which the vehicle 10 is located on the basis of a predetermined traveling route. The horizon may refer to a point at which the vehicle can arrive after a predetermined time from the position at which the vehicle 10 is located along a predetermined traveling route.

The electronic horizon data can include horizon map data and horizon path data.

2.1.1) Horizon Map Data

The horizon map data may include at least one of topology data, road data, HD map data and dynamic data. According to an embodiment, the horizon map data may include a plurality of layers. For example, the horizon map data may include a first layer that matches the topology data, a second layer that matches the road data, a third layer that matches the HD map data, and a fourth layer that matches the dynamic data. The horizon map data may further include static object data.

The topology data may be explained as a map created by connecting road centers. The topology data is suitable for approximate display of a location of a vehicle and may have a data form used for navigation for drivers. The topology data may be understood as data about road information other than information on driveways. The topology data may be generated on the basis of data received from an external server through the communication device 220. The topology data may be based on data stored in at least one memory included in the vehicle 10.

The road data may include at least one of road slope data, road curvature data and road speed limit data. The road data may further include no-passing zone data. The road data may be based on data received from an external server through the communication device 220. The road data may be based on data generated in the object detection device 210.

The HD map data may include detailed topology information in units of lanes of roads, connection information of each lane, and feature information for vehicle localization (e.g., traffic signs, lane marking/attribute, road furniture, etc.). The HD map data may be based on data received from an external server through the communication device 220.

The dynamic data may include various types of dynamic information which can be generated on roads. For example, the dynamic data may include construction information, variable speed road information, road condition information, traffic information, moving object information, etc. The dynamic data may be based on data received from an external server through the communication device 220. The dynamic data may be based on data generated in the object detection device 210.

The processor 170 can provide map data in a range from a position at which the vehicle 10 is located to the horizon.

2.1.2) Horizon Path Data

The horizon path data may be explained as a trajectory through which the vehicle 10 can travel in a range from a position at which the vehicle 10 is located to the horizon. The horizon path data may include data indicating a relative probability of selecting a road at a decision point (e.g., a fork, a junction, a crossroad, or the like). The relative probability may be calculated on the basis of a time taken to arrive at a final destination. For example, if a time taken to arrive at a final destination is shorter when a first road is selected at a decision point than that when a second road is selected, a probability of selecting the first road can be calculated to be higher than a probability of selecting the second road.

The horizon path data can include a main path and a sub-path. The main path may be understood as a trajectory obtained by connecting roads having a high relative probability of being selected. The sub-path can be branched from at least one decision point on the main path. The sub-path may be understood as a trajectory obtained by connecting at least one road having a low relative probability of being selected at at least one decision point on the main path.

3) Control Signal Generation Operation

The processor 170 can perform a control signal generation operation. The processor 170 can generate a control signal on the basis of the electronic horizon data. For example, the processor 170 may generate at least one of a power train control signal, a brake device control signal and a steering device control signal on the basis of the electronic horizon data.

The processor 170 can transmit the generated control signal to the driving control device 250 through the interface 180. The driving control device 250 can transmit the control signal to at least one of a power train 251, a brake device 252 and a steering device 254.

Cabin

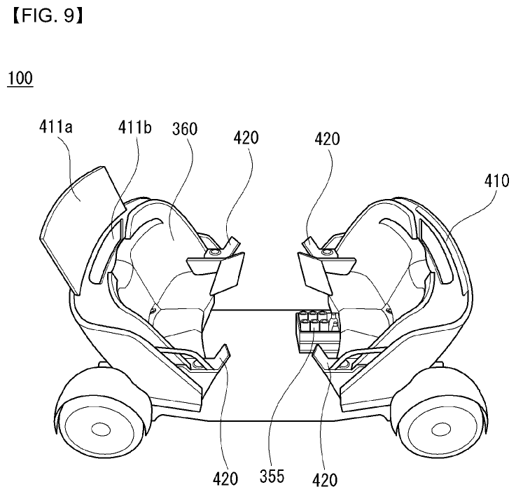

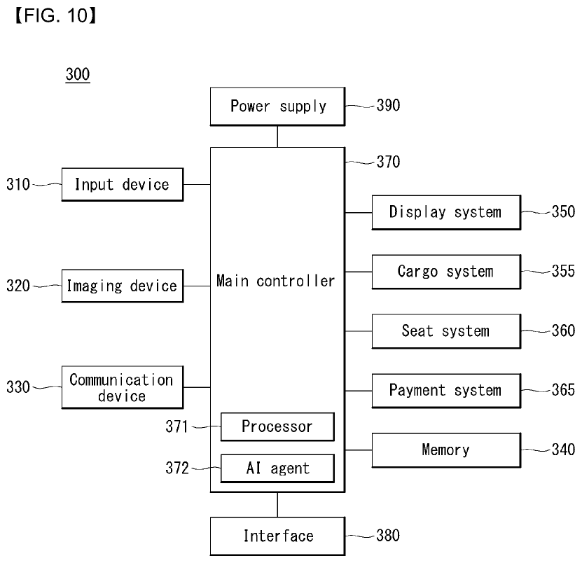

FIG. 9 is a diagram showing the interior of the vehicle according to an embodiment of the present disclosure. FIG. 10 is a block diagram referred to in description of a cabin system for a vehicle according to an embodiment of the present disclosure.

(1) Components of Cabin

Referring to FIGS. 9 and 10, a cabin system 300 for a vehicle (hereinafter, a cabin system) can be defined as a convenience system for a user who uses the vehicle 10. The cabin system 300 can be explained as a high-end system including a display system 350, a cargo system 355, a seat system 360 and a payment system 365. The cabin system 300 may include a main controller 370, a memory 340, an interface 380, a power supply 390, an input device 310, an imaging device 320, a communication device 330, the display system 350, the cargo system 355, the seat system 360 and the payment system 365. The cabin system 300 may further include components in addition to the components described in this specification or may not include some of the components described in this specification according to embodiments.

1) Main Controller

The main controller 370 can be electrically connected to the input device 310, the communication device 330, the display system 350, the cargo system 355, the seat system 360 and the payment system 365 and exchange signals with these components. The main controller 370 can control the input device 310, the communication device 330, the display system 350, the cargo system 355, the seat system 360 and the payment system 365. The main controller 370 may be realized using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, and electronic units for executing other functions.

The main controller 370 may be configured as at least one sub-controller. The main controller 370 may include a plurality of sub-controllers according to an embodiment. The plurality of sub-controllers may individually control the devices and systems included in the cabin system 300. The devices and systems included in the cabin system 300 may be grouped by function or grouped on the basis of seats on which a user can sit.

The main controller 370 may include at least one processor 371. Although FIG. 6 illustrates the main controller 370 including a single processor 371, the main controller 371 may include a plurality of processors. The processor 371 may be categorized as one of the above-described sub-controllers.

The processor 371 can receive signals, information or data from a user terminal through the communication device 330. The user terminal can transmit signals, information or data to the cabin system 300.

The processor 371 can identify a user on the basis of image data received from at least one of an internal camera and an external camera included in the imaging device. The processor 371 can identify a user by applying an image processing algorithm to the image data. For example, the processor 371 may identify a user by comparing information received from the user terminal with the image data. For example, the information may include at least one of route information, body information, fellow passenger information, baggage information, position information, preferred content information, preferred food information, disability information and use history information of a user.

The main controller 370 may include an artificial intelligence (AI) agent 372. The AI agent 372 can perform machine learning on the basis of data acquired through the input device 310. The AI agent 371 can control at least one of the display system 350, the cargo system 355, the seat system 360 and the payment system 365 on the basis of machine learning results.

2) Essential Components

The memory 340 is electrically connected to the main controller 370. The memory 340 can store basic data about units, control data for operation control of units, and input/output data. The memory 340 can store data processed in the main controller 370. Hardware-wise, the memory 340 may be configured using at least one of a ROM, a RAM, an EPROM, a flash drive and a hard drive. The memory 340 can store various types of data for the overall operation of the cabin system 300, such as a program for processing or control of the main controller 370. The memory 340 may be integrated with the main controller 370.

The interface 380 can exchange signals with at least one electronic device included in the vehicle 10 in a wired or wireless manner. The interface 380 may be configured using at least one of a communication module, a terminal, a pin, a cable, a port, a circuit, an element and a device.

The power supply 390 can provide power to the cabin system 300. The power supply 390 can be provided with power from a power source (e.g., a battery) included in the vehicle 10 and supply the power to each unit of the cabin system 300. The power supply 390 can operate according to a control signal supplied from the main controller 370. For example, the power supply 390 may be implemented as a switched-mode power supply (SMPS).

The cabin system 300 may include at least one printed circuit board (PCB). The main controller 370, the memory 340, the interface 380 and the power supply 390 may be mounted on at least one PCB.

3) Input Device

The input device 310 can receive a user input. The input device 310 can convert the user input into an electrical signal. The electrical signal converted by the input device 310 can be converted into a control signal and provided to at least one of the display system 350, the cargo system 355, the seat system 360 and the payment system 365. The main controller 370 or at least one processor included in the cabin system 300 can generate a control signal based on an electrical signal received from the input device 310.

The input device 310 may include at least one of a touch input unit, a gesture input unit, a mechanical input unit and a voice input unit. The touch input unit can convert a user's touch input into an electrical signal. The touch input unit may include at least one touch sensor for detecting a user's touch input. According to an embodiment, the touch input unit can realize a touch screen by integrating with at least one display included in the display system 350. Such a touch screen can provide both an input interface and an output interface between the cabin system 300 and a user. The gesture input unit can convert a user's gesture input into an electrical signal. The gesture input unit may include at least one of an infrared sensor and an image sensor for detecting a user's gesture input. According to an embodiment, the gesture input unit can detect a user's three-dimensional gesture input. To this end, the gesture input unit may include a plurality of light output units for outputting infrared light or a plurality of image sensors. The gesture input unit may detect a user's three-dimensional gesture input using TOF (Time of Flight), structured light or disparity. The mechanical input unit can convert a user's physical input (e.g., press or rotation) through a mechanical device into an electrical signal. The mechanical input unit may include at least one of a button, a dome switch, a jog wheel and a jog switch. Meanwhile, the gesture input unit and the mechanical input unit may be integrated. For example, the input device 310 may include a jog dial device that includes a gesture sensor and is formed such that it can be inserted/ejected into/from a part of a surrounding structure (e.g., at least one of a seat, an armrest and a door). When the jog dial device is parallel to the surrounding structure, the jog dial device can serve as a gesture input unit. When the jog dial device is protruded from the surrounding structure, the jog dial device can serve as a mechanical input unit. The voice input unit can convert a user's voice input into an electrical signal. The voice input unit may include at least one microphone. The voice input unit may include a beam forming MIC.

4) Imaging Device

The imaging device 320 can include at least one camera. The imaging device 320 may include at least one of an internal camera and an external camera. The internal camera can capture an image of the inside of the cabin. The external camera can capture an image of the outside of the vehicle. The internal camera can acquire an image of the inside of the cabin. The imaging device 320 may include at least one internal camera. It is desirable that the imaging device 320 include as many cameras as the number of passengers who can ride in the vehicle. The imaging device 320 can provide an image acquired by the internal camera. The main controller 370 or at least one processor included in the cabin system 300 can detect a motion of a user on the basis of an image acquired by the internal camera, generate a signal on the basis of the detected motion and provide the signal to at least one of the display system 350, the cargo system 355, the seat system 360 and the payment system 365. The external camera can acquire an image of the outside of the vehicle. The imaging device 320 may include at least one external camera. It is desirable that the imaging device 320 include as many cameras as the number of doors through which passengers ride in the vehicle. The imaging device 320 can provide an image acquired by the external camera. The main controller 370 or at least one processor included in the cabin system 300 can acquire user information on the basis of the image acquired by the external camera. The main controller 370 or at least one processor included in the cabin system 300 can authenticate a user or acquire body information (e.g., height information, weight information, etc.), fellow passenger information and baggage information of a user on the basis of the user information.

5) Communication Device

The communication device 330 can exchange signals with external devices in a wireless manner. The communication device 330 can exchange signals with external devices through a network or directly exchange signals with external devices. External devices may include at least one of a server, a mobile terminal and another vehicle. The communication device 330 may exchange signals with at least one user terminal. The communication device 330 may include an antenna and at least one of an RF circuit and an RF element which can implement at least one communication protocol in order to perform communication. According to an embodiment, the communication device 330 may use a plurality of communication protocols. The communication device 330 may switch communication protocols according to a distance to a mobile terminal.

For example, the communication device can exchange signals with external devices on the basis of C-V2X (Cellular V2X). For example, C-V2X may include sidelink communication based on LTE and/or sidelink communication based on NR. Details related to C-V2X will be described later.

For example, the communication device can exchange signals with external devices on the basis of DSRC (Dedicated Short Range Communications) or WAVE (Wireless Access in Vehicular Environment) standards based on IEEE 802.11p PHY/MAC layer technology and IEEE 1609 Network/Transport layer technology. DSRC (or WAVE standards) is communication specifications for providing an intelligent transport system (ITS) service through short-range dedicated communication between vehicle-mounted devices or between a roadside device and a vehicle-mounted device. DSRC may be a communication scheme that can use a frequency of 5.9 GHz and have a data transfer rate in the range of 3 Mbps to 27 Mbps. IEEE 802.11p may be combined with IEEE 1609 to support DSRC (or WAVE standards).

The communication device of the present disclosure can exchange signals with external devices using only one of C-V2X and DSRC. Alternatively, the communication device of the present disclosure can exchange signals with external devices using a hybrid of C-V2X and DSRC.

6) Display System

The display system 350 can display graphic objects. The display system 350 may include at least one display device. For example, the display system 350 may include a first display device 410 for common use and a second display device 420 for individual use.

6.1) Common Display Device

The first display device 410 may include at least one display 411 which outputs visual content. The display 411 included in the first display device 410 may be realized by at least one of a flat panel display, a curved display, a rollable display and a flexible display. For example, the first display device 410 may include a first display 411 which is positioned behind a seat and formed to be inserted/ejected into/from the cabin, and a first mechanism for moving the first display 411. The first display 411 may be disposed such that it can be inserted/ejected into/from a slot formed in a seat main frame. According to an embodiment, the first display device 410 may further include a flexible area control mechanism. The first display may be formed to be flexible and a flexible area of the first display may be controlled according to user position. For example, the first display device 410 may be disposed on the ceiling inside the cabin and include a second display formed to be rollable and a second mechanism for rolling or unrolling the second display. The second display may be formed such that images can be displayed on both sides thereof. For example, the first display device 410 may be disposed on the ceiling inside the cabin and include a third display formed to be flexible and a third mechanism for bending or unbending the third display. According to an embodiment, the display system 350 may further include at least one processor which provides a control signal to at least one of the first display device 410 and the second display device 420. The processor included in the display system 350 can generate a control signal on the basis of a signal received from at last one of the main controller 370, the input device 310, the imaging device 320 and the communication device 330.

A display area of a display included in the first display device 410 may be divided into a first area 411a and a second area 411b. The first area 411a can be defined as a content display area. For example, the first area 411 may display at least one of graphic objects corresponding to can display entertainment content (e.g., movies, sports, shopping, food, etc.), video conferences, food menu and augmented reality screens. The first area 411a may display graphic objects corresponding to traveling situation information of the vehicle 10. The traveling situation information may include at least one of object information outside the vehicle, navigation information and vehicle state information. The object information outside the vehicle may include information on presence or absence of an object, positional information of an object, information on a distance between the vehicle and an object, and information on a relative speed of the vehicle with respect to an object. The navigation information may include at least one of map information, information on a set destination, route information according to setting of the destination, information on various objects on a route, lane information and information on the current position of the vehicle. The vehicle state information may include vehicle attitude information, vehicle speed information, vehicle tilt information, vehicle weight information, vehicle orientation information, vehicle battery information, vehicle fuel information, vehicle tire pressure information, vehicle steering information, vehicle indoor temperature information, vehicle indoor humidity information, pedal position information, vehicle engine temperature information, etc. The second area 411b can be defined as a user interface area. For example, the second area 411b may display an AI agent screen. The second area 411b may be located in an area defined by a seat frame according to an embodiment. In this case, a user can view content displayed in the second area 411b between seats. The first display device 410 may provide hologram content according to an embodiment. For example, the first display device 410 may provide hologram content for each of a plurality of users such that only a user who requests the content can view the content.

6.2) Display Device for Individual Use

The second display device 420 can include at least one display 421. The second display device 420 can provide the display 421 at a position at which only an individual passenger can view display content. For example, the display 421 may be disposed on an armrest of a seat. The second display device 420 can display graphic objects corresponding to personal information of a user. The second display device 420 may include as many displays 421 as the number of passengers who can ride in the vehicle. The second display device 420 can realize a touch screen by forming a layered structure along with a touch sensor or being integrated with the touch sensor. The second display device 420 can display graphic objects for receiving a user input for seat adjustment or indoor temperature adjustment.

7) Cargo System

The cargo system 355 can provide items to a user at the request of the user. The cargo system 355 can operate on the basis of an electrical signal generated by the input device 310 or the communication device 330. The cargo system 355 can include a cargo box. The cargo box can be hidden in a part under a seat. When an electrical signal based on user input is received, the cargo box can be exposed to the cabin. The user can select a necessary item from articles loaded in the cargo box. The cargo system 355 may include a sliding moving mechanism and an item pop-up mechanism in order to expose the cargo box according to user input. The cargo system 355 may include a plurality of cargo boxes in order to provide various types of items. A weight sensor for determining whether each item is provided may be embedded in the cargo box.

8) Seat System

The seat system 360 can provide a user customized seat to a user. The seat system 360 can operate on the basis of an electrical signal generated by the input device 310 or the communication device 330. The seat system 360 can adjust at least one element of a seat on the basis of acquired user body data. The seat system 360 may include a user detection sensor (e.g., a pressure sensor) for determining whether a user sits on a seat. The seat system 360 may include a plurality of seats on which a plurality of users can sit. One of the plurality of seats can be disposed to face at least another seat. At least two users can set facing each other inside the cabin.

9) Payment System

The payment system 365 can provide a payment service to a user. The payment system 365 can operate on the basis of an electrical signal generated by the input device 310 or the communication device 330. The payment system 365 can calculate a price for at least one service used by the user and request the user to pay the calculated price.

(2) Autonomous Vehicle Usage Scenarios

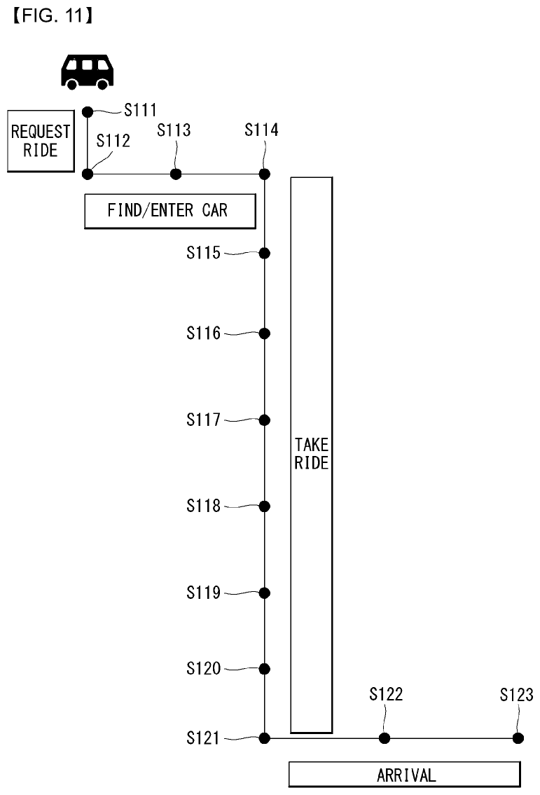

FIG. 11 is a diagram referred to in description of a usage scenario of a user according to an embodiment of the present disclosure.

1) Destination Prediction Scenario

A first scenario S111 is a scenario for prediction of a destination of a user. An application which can operate in connection with the cabin system 300 can be installed in a user terminal. The user terminal can predict a destination of a user on the basis of user's contextual information through the application. The user terminal can provide information on unoccupied seats in the cabin through the application.

2) Cabin Interior Layout Preparation Scenario

A second scenario S112 is a cabin interior layout preparation scenario. The cabin system 300 may further include a scanning device for acquiring data about a user located outside the vehicle. The scanning device can scan a user to acquire body data and baggage data of the user. The body data and baggage data of the user can be used to set a layout. The body data of the user can be used for user authentication. The scanning device may include at least one image sensor. The image sensor can acquire a user image using light of the visible band or infrared band.

The seat system 360 can set a cabin interior layout on the basis of at least one of the body data and baggage data of the user. For example, the seat system 360 may provide a baggage compartment or a car seat installation space.

3) User Welcome Scenario

A third scenario S113 is a user welcome scenario. The cabin system 300 may further include at least one guide light. The guide light can be disposed on the floor of the cabin. When a user riding in the vehicle is detected, the cabin system 300 can turn on the guide light such that the user sits on a predetermined seat among a plurality of seats. For example, the main controller 370 may realize a moving light by sequentially turning on a plurality of light sources over time from an open door to a predetermined user seat.

4) Seat Adjustment Service Scenario

A fourth scenario S114 is a seat adjustment service scenario. The seat system 360 can adjust at least one element of a seat that matches a user on the basis of acquired body information.

5) Personal Content Provision Scenario

A fifth scenario S115 is a personal content provision scenario. The display system 350 can receive user personal data through the input device 310 or the communication device 330. The display system 350 can provide content corresponding to the user personal data.

6) Item Provision Scenario

A sixth scenario S116 is an item provision scenario. The cargo system 355 can receive user data through the input device 310 or the communication device 330. The user data may include user preference data, user destination data, etc. The cargo system 355 can provide items on the basis of the user data.

7) Payment Scenario

A seventh scenario S117 is a payment scenario. The payment system 365 can receive data for price calculation from at least one of the input device 310, the communication device 330 and the cargo system 355. The payment system 365 can calculate a price for use of the vehicle by the user on the basis of the received data. The payment system 365 can request payment of the calculated price from the user (e.g., a mobile terminal of the user).

8) Display System Control Scenario of User

An eighth scenario S118 is a display system control scenario of a user. The input device 310 can receive a user input having at least one form and convert the user input into an electrical signal. The display system 350 can control displayed content on the basis of the electrical signal.

9) AI Agent Scenario

A ninth scenario S119 is a multi-channel artificial intelligence (AI) agent scenario for a plurality of users. The AI agent 372 can discriminate user inputs from a plurality of users. The AI agent 372 can control at least one of the display system 350, the cargo system 355, the seat system 360 and the payment system 365 on the basis of electrical signals obtained by converting user inputs from a plurality of users.

10) Multimedia Content Provision Scenario for Multiple Users

A tenth scenario S120 is a multimedia content provision scenario for a plurality of users. The display system 350 can provide content that can be viewed by all users together. In this case, the display system 350 can individually provide the same sound to a plurality of users through speakers provided for respective seats. The display system 350 can provide content that can be individually viewed by a plurality of users. In this case, the display system 350 can provide individual sound through a speaker provided for each seat.

11) User Safety Secure Scenario

An eleventh scenario S121 is a user safety secure scenario. When information on an object around the vehicle which threatens a user is acquired, the main controller 370 can control an alarm with respect to the object around the vehicle to be output through the display system 350.

12) Personal Belongings Loss Prevention Scenario

A twelfth scenario S122 is a user's belongings loss prevention scenario. The main controller 370 can acquire data about user's belongings through the input device 310. The main controller 370 can acquire user motion data through the input device 310. The main controller 370 can determine whether the user exits the vehicle leaving the belongings in the vehicle on the basis of the data about the belongings and the motion data. The main controller 370 can control an alarm with respect to the belongings to be output through the display system 350.

13) Alighting Report Scenario

A thirteenth scenario S123 is an alighting report scenario. The main controller 370 can receive alighting data of a user through the input device 310. After the user exits the vehicle, the main controller 370 can provide report data according to alighting to a mobile terminal of the user through the communication device 330. The report data can include data about a total charge for using the vehicle 10.

The above-describe 5G communication technology can be combined with methods proposed in the present disclosure which will be described later and applied or can complement the methods proposed in the present disclosure to make technical features of the present disclosure concrete and clear.

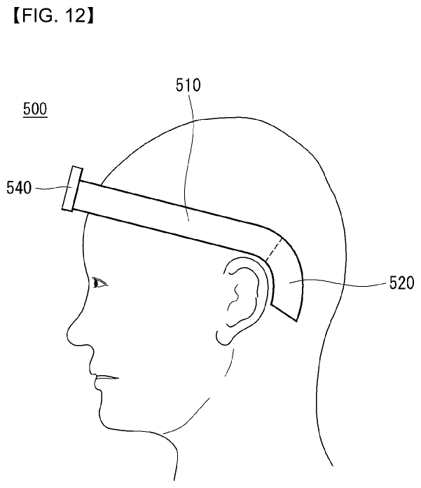

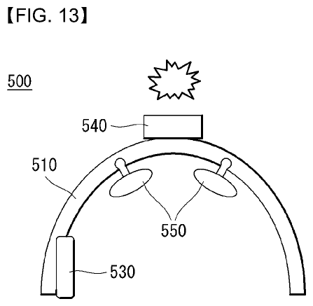

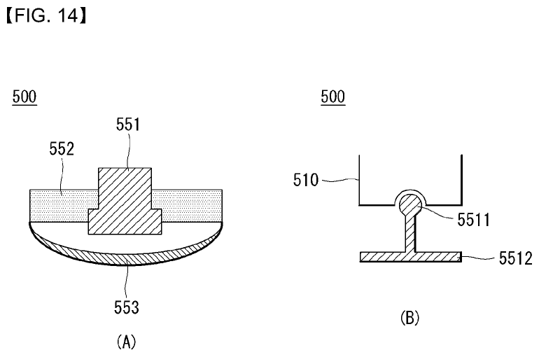

FIGS. 12 to 14 illustrate an EEG detection device according to an embodiment of the present disclosure.

As illustrated in FIGS. 12 to 14, an EEG detection device 500 according to an embodiment of the present disclosure includes a first body 510, a second body 520, and a light output unit 540.

The first body 510 is bent in a round shape so as to come into contact with the forehead of a driver.

An electrode unit 550 for EEG measurement may be provided on a contact surface of the first body 510.

The electrode unit 550 may detect an EEG signal of the driver. The electrode unit 550 may be provided on a forehead contact surface of the first body 510 and a temple contact surface of the first body 510 to detect an EEG signal of the forehead part and the temple part of the driver.

The electrode unit 550 is for measuring a learner's electro-encephalogram (EEG). EEG refers to a signal, which is generated by the brain, measured by the electrode unit 550, and refers to a signal of a fine brain surface which appears as electric signals generated from a number of neurons of the brain are synthesized. The EEG signal changes in time and space according to brain activity, a state of the brain when measured, and a brain function. Thus, EEG signal has a characteristic for each band according to frequencies, a characteristic in a time area, and a spatial characteristic related to a brain function.

Here, the electrode unit 550 is configured in a button shape and may be detachably attached to the first body 510 so as to be replaced. That is, the electrode unit 550 may be connected to the first body 510 through a ball-shaped connection portion rotatable by 360 degrees, and an angle at which the electrode unit 550 is connected to the first body 510 may be appropriately adjusted.

The electrode unit 550 may include a metal 551, a conductive cushion 552, and a conductive cloth 553.

The light output unit 540 may be provided on the other surface of the forehead contact surface of the first body 510 to output predetermined light.

The first body 510 may include a driving module 530 electrically connected to the electrode unit 550. The driving module 530 includes a measuring unit for measuring the EEG signal detected by the electrode unit 550, an analysis unit for analyzing the measured EEG signal, a communication unit transmitting an analysis result from the analysis unit to an external device (e.g., vehicle 10), and a controller controlling the measuring unit, the analysis unit, and the communication unit and controlling the light output unit 540 to output predetermined light. Here, the analysis unit of the driving module 530 may analyze the EEG signal in a digital form and determine a concentration level through an algorithm such as machine learning or the like.

The second body 520 may be electrically connected to the first body 510 and may be electrically connected to an electrode unit (not shown) provided in the first body 510 through the first body 510. When the second body 520 is attached to the driver's skin, the second body 520 may become an electrical reference and ground with respect to a voltage of the electrode unit.

The first body 510 and the second body 520 may be formed of an elastic material (e.g., rubber).

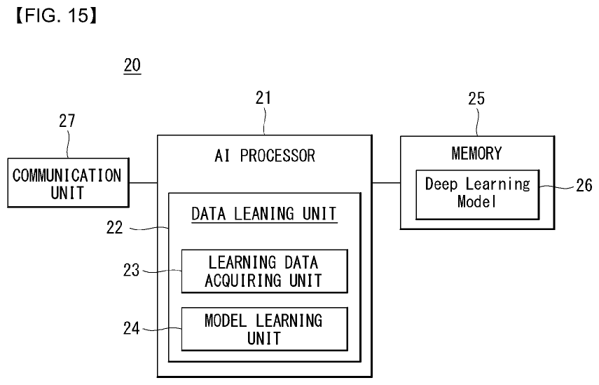

FIG. 15 is a block diagram illustrating an AI device.

The AI device 20 may include an electronic device including an AI module capable of performing AI processing or a server including the AI module. In addition, the AI device 20 may be included in at least a part of the intelligent service providing apparatus 100 illustrated in FIG. 4 and may be provided to perform at least some of the AI processing together.

The AI processing may include all operations related to the control of the intelligent service providing apparatus 100 illustrated in FIG. 4. For example, the intelligent service providing apparatus 100 may AI process the sensing data or the acquired data to perform processing/determination and control signal generation. In addition, for example, the intelligent service providing apparatus 100 may AI process the data received through the communication unit to perform control of the intelligent electronic device.

The AI device 20 may be a client device that directly uses the AI processing result or may be a device in a cloud environment that provides the AI processing result to another device.

The AI device 20 may include an AI processor 21, a memory 25, and/or a communication unit 27.

The AI device 20 is a computing device capable of learning neural networks, and may be implemented as various electronic devices such as a server, a desktop PC, a notebook PC, a tablet PC, and the like.

The AI processor 21 may learn a neural network using a program stored in the memory 25. In particular, the AI processor 21 may learn a neural network for obtaining estimated noise information by analyzing the operating state of each speech providing device. In this case, the neural network for outputting estimated noise information may be designed to simulate the human's brain structure on a computer, and may include a plurality of network nodes having weight and simulating the neurons of the human's neural network. The plurality of network nodes can transmit and receive data in accordance with each connection relationship to simulate the synaptic activity of neurons in which neurons transmit and receive signals through synapses. Here, the neural network may include a deep learning model developed from a neural network model. In the deep learning model, a plurality of network nodes is positioned in different layers and can transmit and receive data in accordance with a convolution connection relationship. The neural network, for example, includes various deep learning techniques such as deep neural networks (DNN), convolutional deep neural networks (CNN), recurrent neural networks (RNN), a restricted boltzmann machine (RBM), deep belief networks (DBN), and a deep Q-network, and can be applied to fields such as computer vision, speech providing, natural language processing, and voice/signal processing.

Meanwhile, a processor that performs the functions described above may be a general purpose processor (e.g., a CPU), but may be an AI-only processor (e.g., a GPU) for artificial intelligence learning.

The memory 25 can store various programs and data for the operation of the AI device 20. The memory 25 may be a nonvolatile memory, a volatile memory, a flash-memory, a hard disk drive (HDD), a solid state drive (SDD), or the like. The memory 25 is accessed by the AI processor 21 and reading-out/recording/correcting/deleting/updating, etc. of data by the AI processor 21 can be performed. Further, the memory 25 can store a neural network model (e.g., a deep learning model 26) generated through a learning algorithm for data classification/recognition according to an embodiment of the present disclosure.

Meanwhile, the AI processor 21 may include a data learning unit 22 that learns a neural network for data classification/recognition. The data learning unit 22 can learn references about what learning data are used and how to classify and recognize data using the learning data in order to determine data classification/recognition. The data learning unit 22 can learn a deep learning model by obtaining learning data to be used for learning and by applying the obtained learning data to the deep learning model.

The data learning unit 22 may be manufactured in the type of at least one hardware chip and mounted on the AI device 20. For example, the data learning unit 22 may be manufactured in a hardware chip type only for artificial intelligence, and may be manufactured as a part of a general purpose processor (CPU) or a graphics processing unit (GPU) and mounted on the AI device 20. Further, the data learning unit 22 may be implemented as a software module. When the data leaning unit 22 is implemented as a software module (or a program module including instructions), the software module may be stored in non-transitory computer readable media that can be read through a computer. In this case, at least one software module may be provided by an OS (operating system) or may be provided by an application.

The data learning unit 22 may include a learning data obtaining unit 23 and a model learning unit 24.

The learning data acquisition unit 23 may obtain training data for a neural network model for classifying and recognizing data. For example, the learning data acquisition unit 23 may obtain an operating state to be input to the neural network model and/or a feature value, extracted from the operating state, as the training data.

The model learning unit 24 can perform learning such that a neural network model has a determination reference about how to classify predetermined data, using the obtained learning data. In this case, the model learning unit 24 can train a neural network model through supervised learning that uses at least some of learning data as a determination reference. Alternatively, the model learning data 24 can train a neural network model through unsupervised learning that finds out a determination reference by performing learning by itself using learning data without supervision. Further, the model learning unit 24 can train a neural network model through reinforcement learning using feedback about whether the result of situation determination according to learning is correct. Further, the model learning unit 24 can train a neural network model using a learning algorithm including error back-propagation or gradient decent.

When a neural network model is learned, the model learning unit 24 can store the learned neural network model in the memory. The model learning unit 24 may store the learned neural network model in the memory of a server connected with the AI device 20 through a wire or wireless network.

The data learning unit 22 may further include a learning data preprocessor (not shown) and a learning data selector (not shown) to improve the analysis result of a recognition model or reduce resources or time for generating a recognition model.

The training data preprocessor may pre-process an obtained operating state so that the obtained operating state may be used for training for recognizing estimated noise information. For example, the training data preprocessor may process an obtained operating state in a preset format so that the model training unit 24 may use obtained training data for training for recognizing estimated noise information.

Furthermore, the training data selection unit may select data for training among training data obtained by the learning data acquisition unit 23 or training data pre-processed by the preprocessor. The selected training data may be provided to the model training unit 24. For example, the training data selection unit may select only data for a syllable, included in a specific region, as training data by detecting the specific region in the feature values of an operating state obtained by the speech providing device 10.

Further, the data learning unit 22 may further include a model estimator (not shown) to improve the analysis result of a neural network model.

The model estimator inputs estimation data to a neural network model, and when an analysis result output from the estimation data does not satisfy a predetermined reference, it can make the model learning unit 22 perform learning again. In this case, the estimation data may be data defined in advance for estimating a recognition model. For example, when the number or ratio of estimation data with an incorrect analysis result of the analysis result of a recognition model learned with respect to estimation data exceeds a predetermined threshold, the model estimator can estimate that a predetermined reference is not satisfied.

The communication unit 27 can transmit the AI processing result by the AI processor 21 to an external electronic device.

Here, the external electronic device may be defined as an autonomous vehicle. Further, the AI device 20 may be defined as another vehicle or a 5G network that communicates with the autonomous vehicle. Meanwhile, the AI device 20 may be implemented by being functionally embedded in an autonomous module included in a vehicle. Further, the 5G network may include a server or a module that performs control related to autonomous driving.

Meanwhile, the AI device 20 shown in FIG. 5 was functionally separately described into the AI processor 21, the memory 25, the communication unit 27, etc., but it should be noted that the aforementioned components may be integrated in one module and referred to as an AI module.

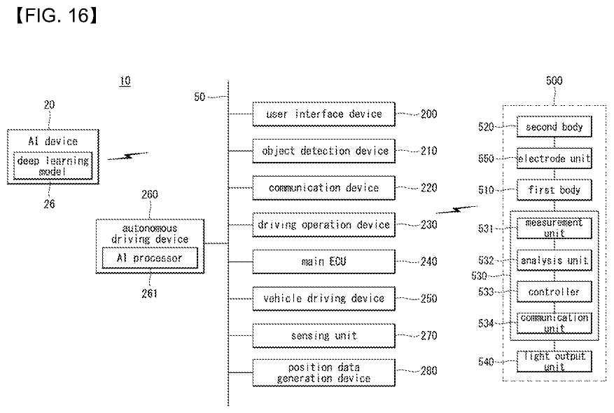

FIG. 16 is a view illustrating a system in which an autonomous vehicle and an artificial intelligence, AI, device are linked according to an embodiment of the present disclosure.

Referring to FIG. 16, the vehicle 10 may transmit data requiring AI processing to the AI device 20 through the communication unit, and the AI device 20 including a deep learning model 26 may transmit an AI processing result using the deep learning model 26 to the vehicle 10. The AI device 20 may refer to the contents described in FIG. 15.

The vehicle 10 may include the components 200, 210, 220, 230, 240, 250, 260, 270, and 280 described above with reference to FIG. 6, and a description of a function performed in the vehicle 10 is the same is the same as that with reference to FIG. 6. Here, the autonomous driving device 260 may further include an AI processor 261.

Hereinafter, another electronic device in the vehicle connected to the user interface device 200 and the AI processor 261 will be described in more detail.

First, the AI processor 261 applies a neural network model to the data obtained through the object detection apparatus 210 to generate at least one of the presence or absence of an object, position information of the object, distance information between the vehicle and the object, and relative speed information between the vehicle and the object.

Meanwhile, the vehicle 10 transmits the data obtained through the at least one sensor to the AI device 20 via the communication device 220, and the AI device 20 applies the neural network model 26 to the transmitted data to generate AI processing data and transmits the AI processing data to the vehicle 10. The vehicle 10 recognizes the information related to an object detected on the basis of the received AI processing data, and the autonomous driving device 260 may perform an autonomous driving control operation using the recognized information.

In the autonomous driving mode, the AI processor 261 may generate an input signal of the driving operation device 230 according to a signal for controlling movement of the vehicle according to a driving plan generated through the autonomous driving device 260.

Meanwhile, the vehicle 10 may transmit data necessary for controlling the driving operation device 230 to the AI device 20 via the communication device 220, and the AI device 20 applies the neural network model 26 to the transmitted data to generate AI processing data and transmits the generated AI processing data to the vehicle 10. The vehicle 10 may use the input signal of the driving operation device 230 for controlling movement of the vehicle on the basis of the received AI processing data.

The driving control device 250 may control a power train, a steering device, and a brake device on the basis of the signal received by the autonomous driving device 260. The signal received by the autonomous driving device 260 may be a driving control signal generated by applying the neural network model to the vehicle-related data in the AI processor 261. The driving control signal may be a signal received from an external AI device 20 via the communication device 220.

The AI processor 261 may generate state data of the vehicle by applying the neural network model to sensing data generated by at least one sensor. The AI processing data generated by applying the neural network model may include vehicle posture data, vehicle motion data, vehicle yaw data, vehicle roll data, vehicle pitch data, vehicle collision data, vehicle direction data, vehicle angle data, vehicle velocity data, vehicle tilt data, vehicle advancing/reversing data, vehicle weight data, battery data, fuel data, tire air pressure data, vehicle interior temperature data, vehicle interior humidity data, steering wheel rotation angle data, vehicle exterior illuminance data, pressure data applied to an accelerator pedal, pressure data applied to the brake pedal, and the like.

The autonomous driving device 260 may generate a driving control signal on the basis of the AI-processed state data of the vehicle.

Meanwhile, the vehicle 10 may transmit sensing data obtained through the at least one sensor to the AI device 20 via the communication unit 22, and the AI device 20 may transmit AI processing data generated by applying the neural network model 26 to the transmitted sensing data to the vehicle 10.

The AI processor 261 may generate more accurate vehicle position data by applying the neural network model to the position data generated by at least one position data generation device.

According to an embodiment, the AI processor 261 may perform a deep learning operation on the basis of at least one of an inertial measurement unit (IMU) of the sensing unit 270 and a camera image of the object detection device 210 and correct the position data on the basis of the generated AI processing data.

Meanwhile, the vehicle 10 transmits the position data obtained from the position data generation device 280 to the AI device 20 via the communication device 220, and the AI device 20 may transmit the AI processing data generated by applying the neural network model 26 to the received position data to the vehicle 10.

The AI processor 261 may transfer a control signal for performing at least one ADAS function described above to the autonomous driving device 260 by applying at least one sensor provided in the vehicle, traffic-related information received from an external device, and information received from another vehicle communicating with the vehicle to the neural network model.

The vehicle 10 may transmit at least data for performing the ADAS functions to the AI device 20 via the communication device 220 and the AI device 20 may transfer a control signal for performing the ADAS function to the vehicle by applying the neural network model 260 to the received data.

The autonomous driving device 260 may obtain the driver's state information and/or vehicle state information through the AI processor 261 and perform an operation of switching from the autonomous driving device 260 performs switching operation from the autonomous driving mode to the manual driving mode or from the manual driving mode to the autonomous driving mode.

Meanwhile, the vehicle 10 may use AI processing data for passenger support, for driving control. For example, as described above, a state of the driver and the occupant may be checked through at least one sensor provided in the vehicle.

Alternatively, the vehicle 10 may recognize a voice signal of the driver or the occupant through the AI processor 261, perform a voice processing operation, and perform a voice synthesis operation.

Hereinabove, the 5G communication required for implementing a vehicle control method and the schematic contents of performing AI processing by applying the 5G communication and transmitting and receiving AI processing results required for implementing the vehicle control method according to an embodiment of the present disclosure have been described.

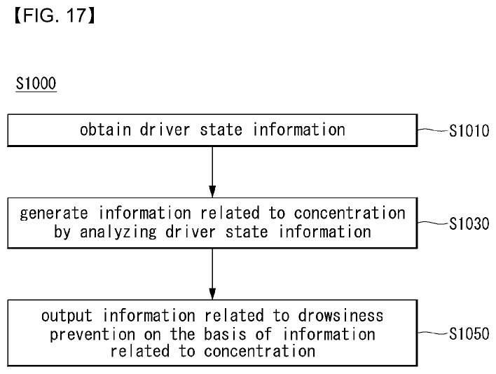

Hereinafter, a specific method of providing information (or interface) for increasing driver's concentration on the basis of driver's state information according to an embodiment of the present disclosure will be described with reference to FIGS. 17 to 27.

FIG. 17 is a flowchart illustrating a vehicle control method according to an embodiment of the present disclosure.

A vehicle control method according to an embodiment of the present disclosure may be implemented in a vehicle including the functions of FIGS. 1 to 16 described above or an intelligent computing apparatus that controls the vehicle. More specifically, the vehicle control method according to an embodiment of the present disclosure may be implemented in the vehicle 10 of FIGS. 1 to 16 described above.

As shown in FIG. 17, a vehicle control method (S1000) according to an embodiment of the present disclosure includes a state information obtaining step (S1010), concentration-related information generating step (S1030), a drowsiness prevention-related information outputting step (S1050), and details thereof are as follows.

First, the autonomous driving device 260 of the vehicle 10 may obtain driver's state information through the sensing unit 270 (S1010).

For example, the sensing unit 270 may include a camera capable of capturing an image of a driver, and the autonomous driving device 260 may obtain the image of the driver captured by the camera. In another example, the sensing unit 270 may include an air sensor capable of detecting a condition of air in the vehicle 10, and the autonomous driving device 260 may obtain a sensing value of the air in the vehicle detected by the air sensor.

For example, the EEG detection device 500 may detect an EEG signal of the driver, and the autonomous driving device 260 may detect an EEG signal detected by the EEG detection device 500 through the communication device 220.

Thereafter, the autonomous driving device 260 may analyze the driver state information and generate information related to the driver's concentration.

For example, the autonomous driving device 260 may determine the driver's concentration as a level of ‘low’, ‘medium’ or ‘high’ and generate information related to concentration indicating that the driver's concentration corresponds to the ‘low’ level, the ‘medium’ level or the ‘high’ level.

Finally, the autonomous driving device 260 may output information related to drowsiness prevention on the basis of the information related to concentration.

According to an embodiment of the present disclosure, information related to drowsiness prevention may be output from the vehicle 10 and may be preset information for preventing the driver from drowning. For example, the information related to drowsiness prevention may include tactile information output through at least one system (e.g., seat system) in the vehicle, as well as visual information such as text information, image information, and the like, including contents of warning against driver's drowsiness and audible information such as a warning sound.

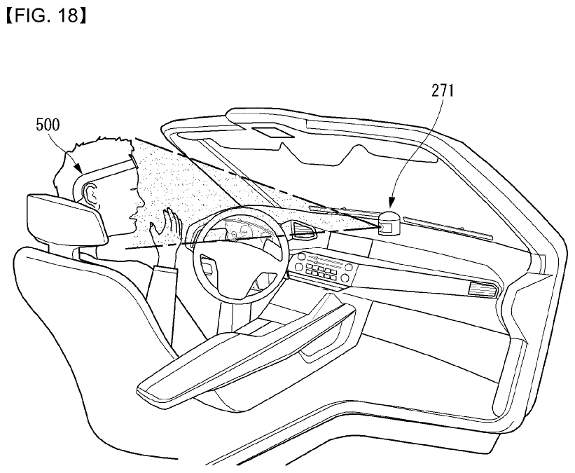

FIG. 18 illustrates an example of obtaining state information of a driver in an embodiment of the present disclosure.

As shown in FIG. 18, the sensing unit of the vehicle 10 may include a camera 271. The camera 271 may capture an image of the driver seated in the vehicle 10 and may transmit the captured image of the driver to the autonomous driving device 260. The autonomous driving device 260 may obtain the driver's image as the driver state information using the data obtained by capturing the driver transmitted from the camera 271.

Also, as illustrated in FIG. 18, the EEG detection device 500 may detect an EEG signal of the driver and transmit the detected EEG signal to the autonomous driving device 260 through the communication unit. The autonomous driving device 260 may obtain the EEG signal of the driver detected by the EEG detection device 500 as driver state information.

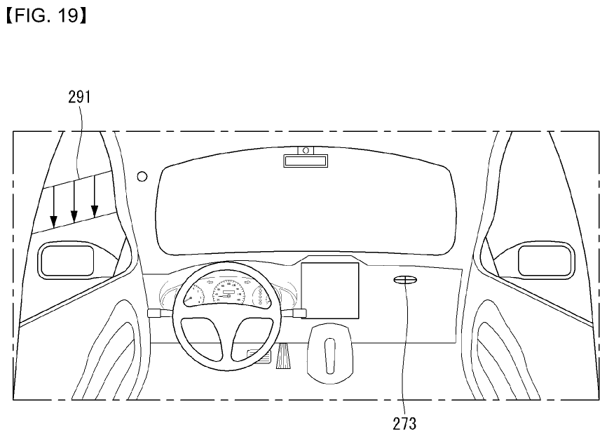

FIG. 19 illustrates an example of outputting information related to drowsiness prevention according to an embodiment of the present disclosure.

According to an embodiment of the present disclosure, the autonomous driving device 260 of the vehicle 10 may obtain driver state information. For example, as shown in FIG. 19, an air sensor 273 may sense a condition of air in the vehicle 10 and transmit the sensed condition of air to the autonomous driving device 260. For example, the air sensor 273 may sense the amount of carbon dioxide in the vehicle 10 and transmit a sensing value related to the sensed amount of carbon dioxide to the autonomous driving device 260.

The autonomous driving device 260 may analyze the driver state information transmitted from the air sensor 273 and other device (e.g., the EEG detection device 500) and determine the driver's concentration level on the basis of the driver state information.

If the driver's concentration level is less than a predetermined threshold value, the autonomous driving device 260 may output information for preventing drowsiness of the driver.

For example, as shown in FIG. 19, if it is determined that the concentration level of the driver is less than the predetermined threshold value, the autonomous driving device 260 may output information related to prevention of the driver's drowsiness. For example, the autonomous driving device 260 may lower (open) the window 291 of the closed vehicle 10.

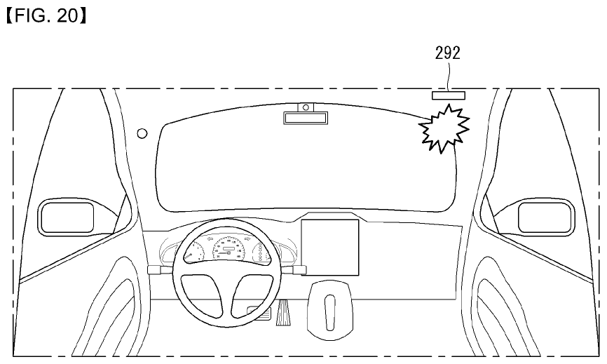

FIG. 20 illustrates another example of outputting information related to drowsiness prevention according to an embodiment of the present disclosure.

According to an embodiment of the present disclosure, the autonomous driving device 260 of the vehicle 10 may determine a concentration level of the driver on the basis of the driver state information, and if the concentration level is less than the threshold value, the autonomous driving device 260 may output information related to prevention of driver's drowsiness in a visual form as described above.

In this connection, as illustrated in FIG. 20, in order to prevent the driver's drowsiness, the autonomous driving device 260 may output visual information related to drowsiness prevention through illumination 292 in the vehicle 10. For example, the autonomous driving device 260 may output light having a specific wavelength through the illumination 292. For example, the autonomous driving device 260 may output lights having different wavelengths through the illumination 292 at predetermined periods.

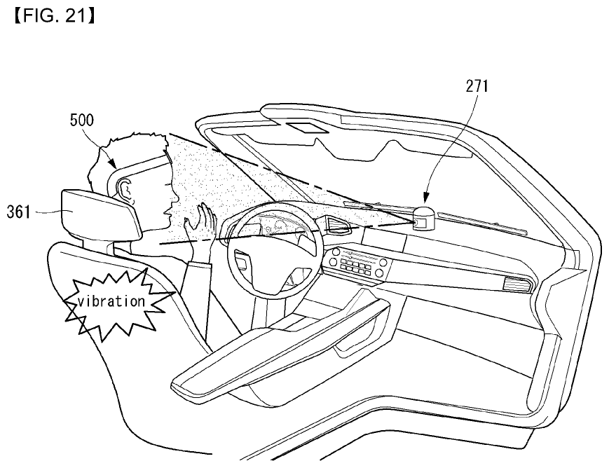

FIG. 21 illustrates another example of outputting information related to drowsiness prevention according to an embodiment of the present disclosure.

According to an embodiment of the present disclosure, the autonomous driving device 260 of the vehicle 10 may determine a concentration level of the driver on the basis of the driver state information, and if the concentration level is less than the threshold value, the autonomous driving device 260 may output information related to prevention of driver's drowsiness in a tactile form.

As shown in FIG. 21, the autonomous driving device 260 may output tactile information related to drowsiness prevention through the seat system 361 in the vehicle 10 to prevent driver's drowsiness. For example, the autonomous driving device 260 may output a vibration effect through the seat system 361. For example, the autonomous driving device 260 may output the vibration effect at a predetermined period through the seat system 361.

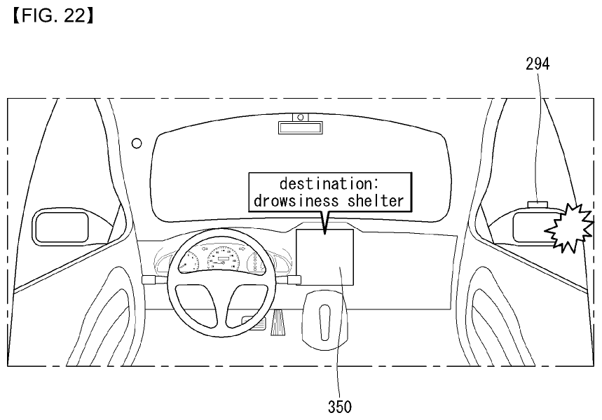

FIG. 22 illustrates another example of outputting information related to drowsiness prevention according to an embodiment of the present disclosure.

According to an embodiment of the present disclosure, the autonomous driving device 260 of the vehicle 10 may determine a concentration level of the driver on the basis of the driver state information, and if the concentration level is less than the threshold value, the autonomous driving device 260 may output information related to prevention of driver's drowsiness in a visual form.

In this connection, as illustrated in FIG. 22, in order to prevent driver's drowsiness, the autonomous driving device 260 may output the image information related to drowsiness prevention through a display system 350 in the vehicle 10. For example, the autonomous driving device 260 may output an image of guiding a route to a specific location (e.g., a drowsiness shelter) in which the driver may take a break through the display system 350.

Also, as illustrated in FIG. 22, the autonomous driving device 260 may output a predetermined light through an emergency light 294 to inform the outside of the vehicle 10 that driver's concentration is less than the threshold value.

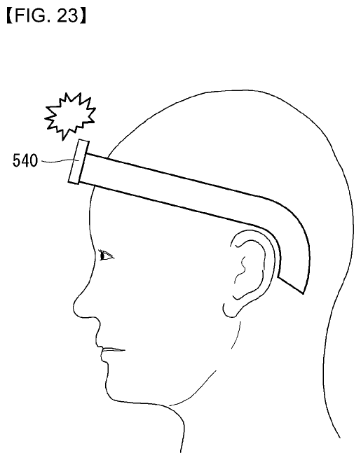

FIG. 23 illustrates another example of outputting information related to drowsiness prevention according to an embodiment of the present disclosure.

According to an embodiment of the present disclosure, the autonomous driving device 260 of the vehicle 10 may determine a concentration level of the driver on the basis of the driver state information, and if the concentration level is less than the threshold value, the autonomous driving device 260 may output information related to prevention of driver's drowsiness in a visual form.

In this connection, as illustrated in FIG. 23, in order to prevent driver's drowsiness, the autonomous driving device 260 may output image information related to drowsiness prevention through the EEG detection device 500 connected to the autonomous driving device 260. For example, the autonomous driving device 260 may control the EEG detection device 500 so that light having a predetermined wavelength is output to the optical output unit 540 of the EEG detection device 500.

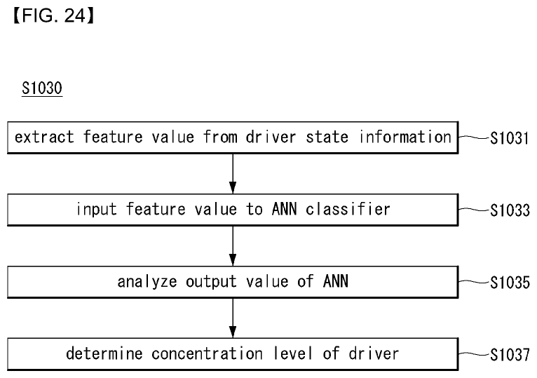

FIG. 24 is a view illustrating an example of determining a concentration level of a driver in an embodiment of the present disclosure.

As shown in FIG. 24, a method of determining a concentration level of a driver (S1030) includes steps S1031 to S1037, and a detailed description thereof will be given below. The concentration level of the driver may be performed by the AI processor 261 in the autonomous driving device 260 or the AI processor 21 of the AI device 20 and the following description is based on the AI processor 261.

First, the AI processor 261 may extract a feature value from the driver state information obtained in step S1010 (S1031). For example, the AI processor 261 may extract a feature value from the EEG signal of the driver detected by the EEG detection device 500.

Next, the AI processor 261 may input the extracted feature value to an artificial neural network (ANN) classifier (S1033). For example, the AI processor 261 may input a feature value of an EEG signal into the ANN classifier.

Thereafter, the AI processor 261 may analyze the output value of the artificial neural network (S1035). For example, the AI processor 261 may obtain a result (output) of inputting the feature value of the EEG signal to the artificial neural network.

Finally, the AI processor 261 may determine a concentration level of the driver on the basis of the result of analyzing the output value of the artificial neural network (S1037).

For example, when the driver takes the following action or when the following situation occurs in the vehicle, if the camera captures corresponding images, the artificial neural network classifier and/or the AI processor 261 may classify the concentration level of the driver as ‘low’.
If the driver uses a mobile phone
If the driver controls a device in the vehicle
If the driver operates navigation
If the driver smokes
If the driver eats food
If the driver watches a video
If the driver views a landscape outside the window
If the driver reads a book or map
If the driver makes up
If the driver is chatting with someone else
If the driver is playing with someone else
If there is a pet in the vehicle
If there is an object that interferes with the driver's gaze
If the driver is looking elsewhere than the front
If an average value of a head position is changed by 10% or more when the driver is driving
If the vehicle enters a curve section
If the vehicle enters a section of frequent accidents
If an average of concentration levels of the accident-free driver who enters a section that the driver entered in the past is equal to or greater than a preset threshold value

Table 1 below illustrates frequency bands according to EEG signals measured by an EEG measuring apparatus, EEG forms and brain states.

TABLE 1

| | Frequency | | |
|---|---|---|---|
| Type of EEG | band | Form of EEG | Brain state |
| Delta | 0.5~4 Hz | [symbol] | Deep sleep |
| Theta | 4~7 Hz | [symbol] | Drowsy, distracted, |
| | | | state of daydream |
| Alpha | 8~12 | [symbol] | External concentration |
| | Hz | | is low in relaxed state |
| Sensory motor | 12~15 | [symbol] | Concentration is |
| rhythm (SMR) | Hz | | maintained |
| | | | in stationary state |
| Beta | 15~18 | [symbol] | Consider idea and |
| | Hz | | maintain concentration |
| | | | in active state |
| High beta | Over 18 | [symbol] | Tense, nervous |
| | Hz | | |

The EEG is classified into delta, theta, alpha, SMR, beta, and high beta according to frequency ranges, and body conditions according to the EEG frequency bands are as shown in Table 1 above.

Delta is 0.5 to 4 Hz and corresponds to deep sleep or brain abnormalities. Theta is 4 to 7 Hz and corresponds to a sleepy or distracted or daydream condition. Alpha is 8 to 12 Hz and corresponds to relaxation or loose concentration state. SMR is 12 to 15 Hz and corresponds to a state of keeping concentration without movement. Beta is 15 to 18 Hz and corresponds to a state of keeping concentration while moving. High beta is 18 Hz and corresponds to a strong stress condition such as tension, anxiety, and irritability.

For example, when the EEG signals detected by the EEG detection device 500 are the same as the example shown in Table 1, the artificial neural network and/or the AI processor 261 may determine a concentration level of the driver on the basis of the states of the brain based on the corresponding EEG signals.

In the meantime, FIG. 24 illustrates the example in which the operation of identifying the concentration level of the driver through the AI processing is implemented in the processing of the vehicle 10, but the present disclosure is not limited thereto. For example, the AI processing may be performed on a 5G network on the basis of sensing information received from the vehicle 10.

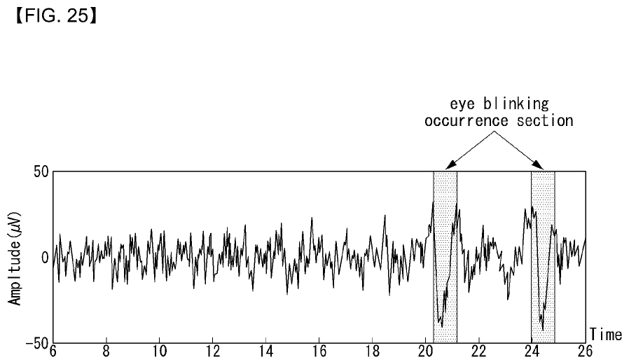

FIG. 25 illustrates an example of EEG changes according to eye movement.

As shown in FIG. 25, when the driver blinks, the EEG signal may change according to the blinking of the driver's eyes.

The AI processor 261 may determine as concentration level of the driver on the basis of the blinking of the driver's eyes.

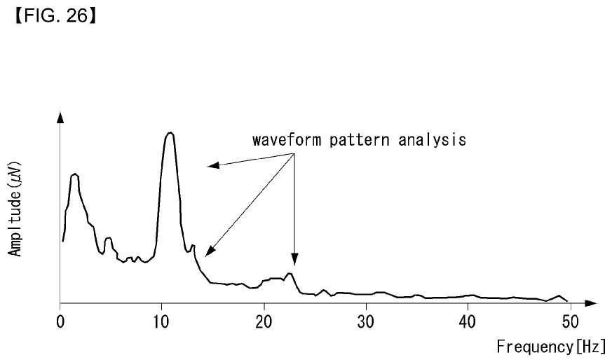

FIG. 26 illustrates an example of EEG modeling including both left/right EEG information and eye movement signals.

The AI processor 261 may determine a concentration level of the driver by analyzing a waveform pattern of the EEG spectrum in consideration of not only left and right brain information of a learner included in the EEG but also a learner's eye movement signal.

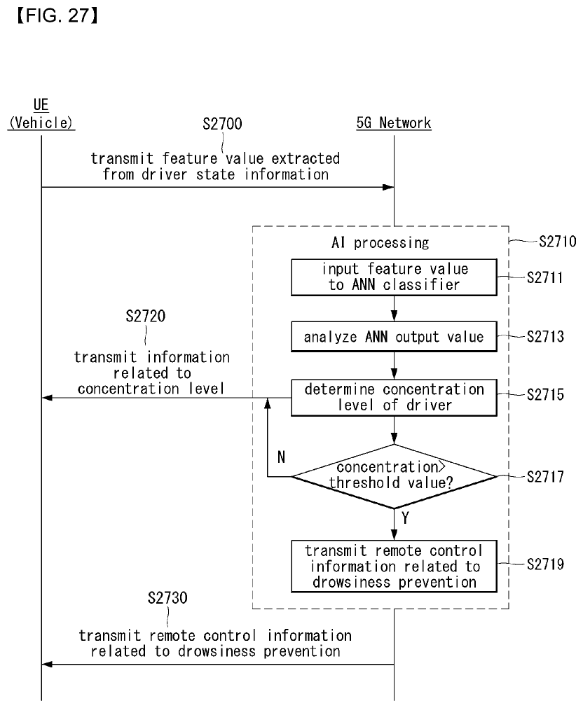

FIG. 27 is a view illustrating another example of determining a drowsy state in an embodiment of the present disclosure.

The processor 170 may control the communication device 220 to transmit state information of the driver to the AI processor included in the 5G network. In addition, the processor 170 may control the communication device 220 to receive AI-processed information from the AI processor.

The AI-processed information may be information indicating a degree (level) of concentration of the driver.

Meanwhile, the vehicle 10 may perform an initial access procedure with the 5G network in order to transmit the state information of the driver to the 5G network. The vehicle 10 may perform the initial access procedure with the 5G network on the basis of a synchronization signal block, SSB.

Also, the vehicle 10 may receive downlink control information (DCI), which is used for scheduling transmission of the state information of the driver obtained from at least one sensor provided in the vehicle through a wireless communication unit, from the network.

The processor 170 may transmit the state information of the driver to the network on the basis of the DCI.

The state information of the driver may be transmitted to the network through a PUSCH, and the SSB and a demodulation reference signal, DM-RS, of the PUSCH may be quasi-co-located, QCL, for a QCL type D.

Referring to FIG. 27, the vehicle 10 may transmit a feature value extracted from the driver state information to the 5G network (S2700).

Here, the 5G network may include an AI processor or an AI system, and the AI system of the 5G network may perform AI processing on the basis of the received sensing information (S2710).

The AI system may input the feature values received from the vehicle 10 to the ANN classifier (S2711). The AI system may analyze the ANN output value (S2713) and determine a concentration level of the driver from the ANN output value (S2715). The 5G network may transmit the concentration level of the driver determined by the AI system to the vehicle 10 through the wireless communication unit.

Here, the concentration level of the driver may include information obtained by digitizing the degree of concentration of the driver or rating information.

In a case where the AI system determines that the concentration of the driver is less than the threshold value (S2717), the AI system may perform drowsiness prevention-related remote control for transmitting information related to drowsiness prevention described above with reference to FIGS. 17 to 27.

If the concentration of the driver is greater than the threshold value, the AI system may determine whether to perform remote control (S2719). In addition, the AI system may transmit information (or signal) related to the remote control to the vehicle 10.

Meanwhile, the vehicle 10 may transmit only the driver state information to the 5G network and extract a feature value corresponding to the driver state information to be used as an input of the artificial neural network for determining the concentration level of the driver form the driver state information in the AI system included in the 5G network.

Meanwhile, the vehicle may interact with at least one robot. The robot may be an autonomous mobile robot (AMR) capable of traveling by magnetic force. The mobile robot may move by itself and may move freely, and a plurality of sensors may be provided to avoid obstacles while driving, and may travel to avoid obstacles. The mobile robot may be a flying robot (eg, a drone) having a flying device. The mobile robot may be a wheeled robot having at least one wheel and moved through rotation of the wheel. The mobile robot may be a legged robot provided with at least one leg and moved using the leg.

The robot may function as a device that supplements the convenience of the vehicle user. For example, the robot may perform a function of moving a load loaded on a vehicle to a user's final destination. For example, the robot may perform a function of guiding a road to a final destination to a user who gets off the vehicle. For example, the robot may perform a function of transporting a user who gets off the vehicle to the final destination.

At least one electronic device included in the vehicle may communicate with the robot through the communication device.

The at least one electronic device included in the vehicle may provide the robot with data processed by the at least one electronic device included in the vehicle. For example, the at least one electronic device included in the vehicle may provide at least one of object data, HD map data, vehicle state data, vehicle location data, and driving plan data to the robot.

At least one electronic device included in the vehicle may receive data processed by the robot from the robot. The at least one electronic device included in the vehicle may receive at least one of sensing data generated by the robot, object data, robot state data, robot position data, and movement plan data of the robot.

At least one electronic device included in the vehicle may generate a control signal based on data received from the robot. For example, the at least one electronic device included in the vehicle may compare the information about the object generated in the object detecting apparatus with the information about the object generated by the robot, and generate a control signal based on the comparison result. Can be. At least one electronic device included in the vehicle may generate a control signal so that interference between the movement path of the vehicle and the movement path of the robot does not occur.

At least one electronic device included in the vehicle may include a software module or a hardware module (hereinafter, referred to as an artificial intelligence module) that implements artificial intelligence (AI). The at least one electronic device included in the vehicle may input the obtained data into the artificial intelligence module and use the data output from the artificial intelligence module.

The artificial intelligence module may perform machine learning on input data using at least one artificial neural network (ANN). The artificial intelligence module may output driving plan data through machine learning on input data.

At least one electronic device included in the vehicle may generate a control signal based on data output from the artificial intelligence module.

According to an embodiment, at least one electronic device included in the vehicle may receive data processed by an artificial intelligence from an external device through a communication device. At least one electronic device included in the vehicle may generate a control signal based on data processed by artificial intelligence.

The present invention described above can be embodied as computer readable codes on a medium in which a program is recorded. The computer-readable medium includes all kinds of recording devices in which data that can be read by a computer system is stored. Examples of computer-readable media include hard disk drives (HDDs), solid state disks (SSDs), silicon disk drives (SDDs), ROMs, RAMs, CD-ROMs, magnetic tapes, floppy disks, optical data storage devices, and the like. This also includes implementations in the form of carrier waves (eg, transmission over the Internet). Accordingly, the above detailed description should not be construed as limiting in all aspects and should be considered as illustrative. The scope of the invention should be determined by reasonable interpretation of the appended claims, and all changes within the equivalent scope of the invention are included in the scope of the invention.

1. A method for controlling a vehicle, the method comprising:
obtaining state information related to a driver in a vehicle; and
outputting information related to drowsiness prevention on the basis of the state information related to the driver,
wherein the outputting the information related to drowsiness prevention comprises:
generating information related to concentration of the driver by analyzing the state information related to the driver; and
outputting the information related to drowsiness prevention on the basis of the information related to concentration.

2. The method of claim 1, wherein
the obtaining of the state information related to the driver comprises detecting an electro-encephalography, EEG, signal of the driver.

3. The method of claim 1, wherein
the obtaining the state information related to the driver comprises capturing an image of the driver, and
the generating of the information related to concentration of the driver comprises generating the information related to concentration on the basis of action information of the captured image of the driver.

4. The method of claim 1, wherein
the outputting the information related to drowsiness prevention comprises outputting light having a predetermined wavelength through illumination of the vehicle.

5. The method of claim 1, wherein
the outputting of the information related to drowsiness prevention comprises outputting vibration through a seat of the vehicle.

6. The method of claim 1, wherein
the outputting of the information related to drowsiness prevention comprises outputting information for guiding a road to a predetermined rest area through a display of the vehicle.

7. The method of claim 1, further comprising:
receiving, from a network, downlink control information (DCI) used for scheduling transmission of state information of the driver obtained from at least one sensor provided in the vehicle,
wherein the state information of the driver is transmitted to the network on the basis of the DCI.

8. The method of claim 7, further comprising:
performing an initial access procedure with the network on the basis of a synchronization signal block, SSB,
wherein the state information of the driver is transmitted to the network through a PUSCH, the SSB and a DM-RS of the PUSCH are quasi-co-located, QCL, for a QCL type D.

9. The method of claim 8, further comprising:
transmitting the state information of the driver to an artificial intelligence, AI, processor included in the network; and
receiving AI-processed information from the AI processor,
wherein the AI processed information is information determined as one of a low concentration state (low), a medium concentration state (medium), and a high concentration state (high).

10. An intelligent computing apparatus for controlling a vehicle, the intelligent computing apparatus comprising:
a camera included in the vehicle;
a sensing unit;
a processor; and
a memory including an instruction executable by the processor,
wherein the instruction enables obtaining of state information related to a driver in the vehicle and outputting information related to drowsiness prevention on the basis of the state information related to the driver, and
the outputting of the state information related to the driver comprises:
generating information related to concentration of the driver by analyzing the state information related to the driver and
outputting the information related to drowsiness prevention on the basis of the information related to concentration.

11. The intelligent computing apparatus of claim 10, wherein
the processor detects an electro-encephalography, EEG, signal of the driver.

12. The intelligent computing apparatus of claim 10, wherein
the processor captures an image of the driver and generates the information related to concentration on the basis of action information of the captured image of the driver.

13. The intelligent computing apparatus of claim 10, wherein
the processor outputs light having a predetermined wavelength through illumination of the vehicle.

14. The intelligent computing apparatus of claim 10, wherein
the processor outputs vibration through a seat of the vehicle.

15. The intelligent computing apparatus of claim 10, wherein
the processor outputs information guiding a route to a predetermined rest area through a display of the vehicle.

16. The intelligent computing apparatus of claim 10, wherein
the processor receives, from a network, downlink control information (DCI) used for scheduling transmission of state information of the driver obtained from at least one sensor provided in the vehicle, and the state information of the driver is transmitted to the network on the basis of the DCI.

17. The intelligent computing apparatus of claim 16, wherein
the processor performs an initial access procedure with the network on the basis of a synchronization signal block, SSB,
the state information of the driver is transmitted to the network through a PUSCH, and
the SSB and a DM-RS of the PUSCH are quasi-co-located, QCL, fora QCL type D.

18. The intelligent computing apparatus of claim 17, wherein
the processor controls a communication unit to transmit the state information of the driver to a AI processor included in the network and control the communication unit to receive the AI-processed information from the AI processor, and
the AI-processed information is information determined as one of a low concentration state (low), a medium concentration state (medium), and a high concentration state (high).
